(19) United States
(12) Patent Application Publication
(10) Pub. No.: US 2018/0371046 A1
(43) Pub. Date: Dec. 27, 2018

(54) Therapy for Lower Urinary Tract Dysfunctions

(71) Applicant: University of Pittsburgh - Of the Commonwealth System of Higher Education, Pittsburgh, PA (US)

(72) Inventors: Anthony J. Kanai, Pittsburgh, PA (US); Guy Salama, Pittsburgh, PA (US); Irina Zabbarova, Pittsburgh, PA (US); Youko Ikeda, Pittsburgh, PA (US); Bethann Weber, Pittsburgh, PA (US)

(21) Appl. No.: 15/967,986

(22) Filed: May 1, 2018

Related U.S. Application Data
(60) Provisional application No. 62/492,498, filed on May 1, 2017.

(51) Int. Cl. C07K 14/64; A61K 9/00; C07K 14/65; A61K 38/22; A61P 13/02

(52) U.S. Cl. CPC C07K 14/64; A61K 9/0019; A61P 13/02; A61K 38/2221; C07K 14/65

(57) ABSTRACT

The present invention relates to the use of relaxin for the treatment of lower urinary tract dysfunctions.

### CROSS-REFERENCE TO RELATED APPLICATIONS

This application claims the benefit of U.S. Provisional Patent Application No. 62/492,498 filed on May 1, 2017, which is incorporated herein by reference in its entirety.

### STATEMENT REGARDING FEDERALLY SPONSORED RESEARCH

This invention was made with government support under Grant No. DK071085, awarded by the National Institutes of Health. The government has certain rights in the invention.

The Sequence Listing associated with this application is filed in electronic format via EFS-Web and is hereby incorporated by reference into the specification in its entirety. The name of the text file containing the Sequence Listing is 65271802393_ST25.txt. The size of the text file is 5,700 bytes, and the text file was created on Apr. 19, 2018.

### BACKGROUND OF THE INVENTION

### Field of the Invention

Methods of treatment of lower urinary tract dysfunction are provided.

### Description of Related Art

Radiation Cystitis can result from radiation therapy for pelvic organ tumors in men and women that are estimated to account for 35% and 18%, respectively, of new malignancies diagnosed in the United States in 2017, according to the American Cancer Society (Siegel, R. et al.: Cancer statistics, 2018. CA Cancer J Clin, 68: 7, 2018). While irradiation is a key therapy for treating these malignancies, the radiation dose is limited by the potential for developing radiation cystitis. Accordingly, radiation therapy is typically fractionated into daily 0.5-2 Gray (1 Gy=100 Rads) increments until the desired dose is achieved (e.g., 20-60 Gy) (Petrovich, Z., et al.: Radiotherapy for carcinoma of the bladder: a review. Am J Clin Oncol, 24: 1, 2001).

The acute symptoms of radiation cystitis can occur within days and may include bladder inflammation, urgency, frequency, dysuria and incontinence (Browne, C., et al.: A Narrative Review on the Pathophysiology and Management for Radiation Cystitis. Adv Urol, 2015). Initiation factors are disruption of the urothelial cell permeability barrier and sensitization of afferent nerves (Crowe, R., et al.: Radiation-induced changes in neuropeptides in the rat urinary bladder. J Urol, 156: 2062, 1996). In addition to DNA damage, irradiation induces Ca²⁺ influx and release in urothelial cells that activate nitric oxide synthases. Nitric oxide (NO.), in turn, binds to cytochrome oxidase which can inhibit the mitochondrial respiratory chain. This results in the production of superoxide (.O₂⁻) which reacts with NO to form peroxynitrite (ONO₂⁻) which further inhibits respiration and damages proteins leading to swelling and rupture of the mitochondria, cytochrome c release and urothelial cell apoptosis (Zabbarova, I., et al.: Targeted delivery of radioprotective agents to mitochondria. Mol Interv, 8: 294, 2008). The leading candidates responsible for this Ca²⁺ influx are transient receptor potential ankyrin 1 and vanilloid 1 (TRPA1 and TRPV1) channels that are highly expressed in urothelial cells (Streng, T., et al.: Distribution and function of the hydrogen sulfide-sensitive TRPA1 ion channel in rat urinary bladder. Eur Urol, 53: 391, 2008, and Birder, L. A., et al.: Altered urinary bladder function in mice lacking the vanilloid receptor TRPV1. Nat Neurosci, 5: 856, 2002). TRPA1 is known to be activated by acrolein, a byproduct of fatty acid lipid peroxidation generated in cells by ionizing radiation (Uchida, K., et al.: Acrolein is a product of lipid peroxidation reaction. Formation of free acrolein and its conjugate with lysine residues in oxidized low density lipoproteins. J Biol Chem, 273: 16058, 1998). Activation of TRPA1 by acrolein in urothelial cells (and TRPV1 by capsaicin) results in chemical cystitis with symptomology similar to radiation cystitis (Bjorling, D. E., et al.: Acute acrolein-induced cystitis in mice. BJU Int, 99: 1523, 2007) as schematized in FIG. 1A. Moreover, these channels are highly expressed in mast cells and in sensory nerves innervating the bladder, colon and other pelvic organs, which suggests that irradiation may sensitize afferent nerves (FIG. 1B). The most radiosensitive cells in the body include lymphocytes and hematopoietic cells of the immune system, the capillary endothelial cells of the gastrointestinal (GI) tract and the urothelial cells of the urinary bladder (Zabbarova, I., et al.: Mol Interv, 8: 294, 2008). Therefore, whole body irradiation >10 Gy can be lethal due to a breakdown of the GI tract and urinary bladder barriers in the presence of a compromised immune response.

Chronic radiation cystitis can develop within 6-12 months, with its prevalence reaching ˜7% (Smit, S. G., et al.: Management of radiation cystitis. Nat Rev Urol, 7: 206, 2010). The symptoms include vascular endothelial cell damage, ischemia, collagen deposition and decreased bladder compliance. The main presenting feature for the chronic phase is hematuria which can range from mild to life-threatening and may include urinary retention, secondary to clots obstructing the urethra and severely decreased bladder compliance due to collagen deposition (Smit, S. G., et al.: Nat Rev Urol, 7: 206, 2010) (FIG. 1C), which may result in an acontractile detrusor requiring a cystectomy. Therapy includes transurethral catheterization with bladder washout and irrigation, laser fulguration, electrocoagulation, pentosan polysulfate and hyperbaric oxygen therapy (Browne, C., et al.: A Narrative Review on the Pathophysiology and Management for Radiation Cystitis. Adv Urol, 2015: 346812, 2015). These chronic approaches are invasive, often fail to demonstrate optimal efficacy, and do not improve bladder compliance for which there is currently no effective treatment and which remains an unmet public health problem.

In addition to radiation cystitis, collagen deposition and decreased bladder compliance can occur with aging, leading to bladder underactivity/underactive bladder (UAB) syndrome. It should be noted that a majority of the elderly may initially exhibit bladder overactivity/overactive bladder (OAB) syndrome for which there are a number of effective therapies including muscarinic receptor blockers, β₃-adrenergic receptor agonists, botulinum neurotoxin type-A and various neuromodulation approaches (Osman, N. I., et al.: Overactive bladder syndrome: Current pathophysiological concepts and therapeutic approaches. Arab J Urol, 11: 313, 2013). However, over time, overactivity commonly reverts to underactivity for which there are currently no effective treatments.

Bladder underactivity, according to the International Continence Society, is a contraction of reduced strength and/or duration, resulting in prolonged voiding and/or failure to achieve complete bladder emptying within a normal time span based on a urodynamic diagnosis. On the other hand, UAB syndrome covers the general condition irrespective of whether the cause is afferent dysfunction, lack of CNS control, or the detrusor itself (Osman, N. I., et al.: Detrusor underactivity and the underactive bladder: a new clinical entity? A review of current terminology, definitions, epidemiology, aetiology, and diagnosis. Eur Urol, 65: 389, 2014, and Andersson, K. E. The many faces of impaired bladder emptying. Curr Opin Urol, 24: 363, 2014). There are several animal models for studying bladder underactivity/UAB (Tyagi, P., et al.: Pathophysiology and animal modeling of underactive bladder. Int Urol Nephrol, 46 Suppl 1: S11, 2014) including: type I and II diabetic mice and rats; partial bladder outlet obstruction (PBOO) and bladder overdistension; ischemia/reperfusion and oxidative stress (due to H₂O₂ instillation); pelvic nerve cut and crush; and aging typically using 18-24 month old rats. While aging is perhaps the most physiologically relevant of these models, it is also one of the most difficult to study because of survival, variability and cost issues. Accordingly, in the United States, rats for these studies are typically obtained by qualified investigators through the National Institute on Aging (NIA) of the National Institutes of Health (NIH).

Relaxin is a 6 kDa hormone, first described in 1926 (Hisaw, F. L. Experimental relaxation of the pubic ligament of the guinea pig. Exp Biol Med (Maywood), 23: 661, 1926), that is produced mainly by the corpus luteum in the ovaries to relax the uterus and soften the pubic symphysis during pregnancy (Bathgate, R. A., et al.: Relaxin family peptides and their receptors. Physiol Rev, 93: 405, 2013), but is also produced in the prostate and testes to enhance sperm motility (Lessing, J. B., et al.: Effect of relaxin on human spermatozoa. J Reprod Med, 31: 304, 1986). It belongs to the insulin superfamily with 7 members exhibiting high structural but low sequence homology; relaxin-1, relaxin-2, and relaxin-3 (RLX1, RLX2, and RLX3, respectively) and insulin-like peptide-3, insulin-like peptide-4, insulin-like peptide-5, and insulin-like peptide-6 (Halls, M. L., et al.: International Union of Basic and Clinical Pharmacology. XCV. Recent advances in the understanding of the pharmacology and biological roles of relaxin family peptide receptors 1-4, the receptors for relaxin family peptides. Pharmacol Rev, 67: 389, 2015). It is formed as a three chain pro-hormone, cleaving off one of the chains to form the active heterodimer with 24 and 29 amino acids linked by disulfide bridges. RLX1 and RLX2 are both circulating proteins which bind the same receptors to downregulate inflammation, modulate autoimmunity, and assist in neuronal function and vasodilatation. They differ in their regulation: RLX1 is present at all times in the body, and is up-regulated by stress and the inflammatory response, RLX2 production is up-regulated by estradiol and growth hormones. Unlike RLX1 and RLX2, RLX3 is produced in the brain and is classified as a neurotransmitter.

Human insulin-like peptide-3, a peptide also included in the insulin superfamily of peptides, is a protein that in humans is encoded by the INSL3 gene. The protein encoded by this gene is an insulin-like hormone produced mainly in gonadal tissues in males and females. Studies of the mouse counterpart suggest that this gene may be involved in the development of urogenital tract and female fertility. It may also act as a hormone to regulate growth and differentiation of gubernaculum thus mediating intra-abdominal testicular descent. Mutations in this gene may lead to cryptorchidism. Human relaxin-1 (hRLX1) and human relaxin-2 (hRLX2) bind preferentially to relaxin receptor 1 and human insulin-like peptide-3 (hINSL3) binds preferentially to relaxin receptor 2.

Insulin superfamily peptides are pleiotropic proteins and have the ability to influence multiple pathways. There are four G-protein coupled receptors (Relaxin Family Peptide Receptors 1-4, or RXFP1-4) which the insulin superfamily peptides bind to. Out of these, RXFP1 is the most studied in humans and rodents and for which human relaxin-2 has the highest affinity. These receptors have broad distribution (Bathgate, R. A., et al.: Physiol Rev, 93: 405, 2013), including smooth muscle, connective tissue, the nervous system, heart and, as shown here, the urinary bladder.

### SUMMARY

Described herein are methods of treating lower urinary tract dysfunctions by administering to a patient in need thereof an amount of a relaxin, e.g. a human insulin superfamily peptide, for instance, a human relaxin-2 peptide effective to treat the lower urinary tract dysfunction. Relaxin is shown herein to be an effective and safe therapy for the treatment of urinary tract diseases such as radiation cystitis and the consequence of ‘normal’ aging. Relaxin also is shown herein to be effective in the treatment of lower urinary tract dysfunctions resulting from benign prostate hyperplasia, interstitial and chemical (e.g., cyclophosphamide and ketamine) induced cystitis/nephritis (see, FIG. 2).

### BRIEF DESCRIPTION OF THE DRAWINGS

FIG. 1. Mechanisms for development of radiation cystitis following exposure to ionizing radiation. A. Urothelial cells are one of the most susceptible to irradiation damage. Oxidative byproducts activate TRPA1 and TRPV1 channels to cause a sustained intracellular Ca²⁺ rise, activation of nitric oxide synthase (NOS), inhibition of the mitochondrial respiratory chain and, as a consequence, generation of nitrogen and oxygen free radicals. This sequence of events leads to the initiation of apoptotic pathways, disruption of the urothelial barrier and further inflammation in the bladder wall. B. TRPA/V1 channels are present on sensory neurons innervating the bladder and these could also be activated to cause neuropeptide release and afferent sensitization observed in acute stages of radiation cystitis. Furthermore, mast cell TRPA1 channel activation may induce degranulation and release of inflammatory mediators. C. The self-perpetuating inflammation initiated by ionizing radiation leads to collagen deposition throughout the lamina propria and between muscle cells in the detrusor layer. This causes decreased detrusor contractility and the reduced bladder compliance and capacity observed in chronic stages of radiation cystitis.

FIG. 2. Schematic of proposed therapeutic effects of relaxin on fibrosis and upper and lower urinary tract dysfunctions. Aging, cystitis and nephritis can contribute to inflammatory processes leading to collagen deposition and decreased detrusor and kidney functions. Chronically, these can result in bladder underactivity and renal failure.

FIG. 3. Putative Mechanism of Action of hRLX2. A. Aging results in increasing levels of reactive nitrogen and oxygen species leading to recurrent bouts of inflammation, elevated levels of TGFβ-1 and fibrosis of the bladder wall. B. Disruption of the urothelium (UT) causes recruitment/activation of immune cells that generate an inflammatory response. C. Inflammatory cytokines and growth factors including TGFβ-1 are released and cause transition of fibroblasts to myofibroblasts increasing collagen synthesis and extracellular matrix (ECM deposition (i.e., fibrosis). The hRLX2 hormone (SEQ ID NOs: 4 and 5) is capable of binding to RXFP1/2 receptors located on immune cells, myofibroblasts and smooth muscle cells. D. Activation of RXFP1 and RXFP2 by hRLX2 can suppress pro-inflammatory and pro-fibrotic processes while enhancing ECM degradation and muscle contractile responses, via increases in Cav1.2. The combined effect reverses irradiation- and aging-induced inflammation and fibrosis to improve bladder function.

FIG. 4. Cystometric and EUS electromyographic assessment of irradiated mice with and without relaxin treatment. A. Selective bladder irradiation model. B-D. Bladder and sphincter functions were assessed using decerebrated cystometry with simultaneous electromyography (EMG; green traces) at 9 weeks following bladder irradiation. Relaxin administration abolished overflow incontinence with a return of voiding function and improved bladder compliance (D) compared to untreated irradiated animals (C). Normal sphincter activity (B; right panel) demonstrated the guarding reflex with an increase in sphincter tension and bladder pressure just before bursting occurs during which tonic activity decreases and voiding occurs. Irradiated mice exhibited a prolonged guarding reflex without bursting (C) which recovered with relaxin treatment (D).

FIG. 5A-5D. Relaxin treatment improves detrusor contractility and reduces bladder fibrosis in mouse bladders with chronic stage radiation cystitis. FIG. 5A. Length-tension profiles from irradiated (9-weeks post exposure) mouse bladders with and without relaxin treatment. Chronic radiation cystitis caused a significant increase in passive tension and decrease in active tension which was reversed by relaxin treatment to a profile similar to non-irradiated control mouse bladders. FIG. 5B. Relaxin treatment increased the contractile response of the detrusor to muscarinic receptor agonist, oxotremorine-M. There was no significant change in responses to purinergic agonist (ABMA), depolarization (KCl) or electrical field stimulation (EFS). FIG. 5C. Histological examination of irradiated mouse bladders showed a minimal urothelial layer and increases in the thickness of the lamina propria and overall collagen content. Relaxin treated irradiated bladders had an intact urothelium, normal lamina propria and increased thickness of the detrusor layer. Expression of the L-type Ca²⁺ channel subunit, CaV_{1.2}, was examined by immunohistochemistry. CaV_{1.2} labeling (green fluorescence, blue; DAPI nuclear stain) in the detrusor was found to be nearly absent in irradiated bladders and was returned by relaxin treatment. FIG. 5D. Quantification of collagen content from sham and relaxin treated irradiated mouse bladders showed a significant decrease in collagen content following relaxin treatment.

FIG. 6A-6D. Relaxin treatment reduces age-related bladder fibrosis in aged rats. FIG. 6A. Length-tension profiles from bladders of aged (>24 months, red and blue lines) and adult (9 months, green and black lines) rats demonstrated that aged animals had significantly stiffer bladders as seen from the passive tension profile. Treatment with relaxin (saline given for sham controls) decreased the passive tension profile to that of adult animals. There were no significant changes in active tension generation between all groups. FIG. 6B. The lack of change in active tension profiles was also matched by no significant change in responses to contractile agonists (ABMA and oxotremorine-M) or depolarization (KCl and EFS). FIG. 6C. Quantification of collagen to tissue ratio in aged and adult rat bladders demonstrated a significant decrease in collagen content following relaxin treatment in aged rats, but not in adults. FIG. 6D. Example tissue sections with collagen staining in aged and adult rat bladders with and without relaxin treatment. In 9 month old animals, the size of each bladder layer and collagen distribution was not significantly different between sham and relaxin treated groups. In aged sham rats, the detrusor layer was thinner compared to 9-month animals and proportionally there was more collagen. Bladders from relaxin treated aged rats appeared to have increased detrusor layer thickness and less collagen infiltration compared to shams.

FIG. 7. Expression of L-type Ca²⁺ channels is increased in aged rat bladders following relaxin treatment. Immunohistological staining of aged (>24 month old) rat bladder sections for the L-type Ca²⁺ channel al C subunit, Cav1.2α. Expression of Cav1.2α was increased in relaxin-treated rat bladders compared to sham controls, similar to that observed in irradiated mouse bladders (FIG. 5C). The expression of Cav1.2α was patchy in aged rat bladders and did not appear to be completely absent as seen with the bladders of the irradiation model. This emphasizes the heterogeneity of aging related bladder changes.

FIG. 8. Changes in bladder compliance demonstrated in length-tension curves. In aged rats, passive tension began increasing at a smaller degree of stretch compared to relaxin treated animals. Active tension profiles were equivocal between relaxin and vehicle treated aged rat bladders. There was no significant difference in the passive tension profile of relaxin versus vehicle treated adult rat bladders. However, relaxin treated adult rat bladders showed increased active tension generation compared to controls.

FIG. 9. Collagen deposition in aged bladders and its reversal with relaxin. Representative histological bladder sections are shown in this figure from 9 month adult and 24 month aged male rats administered hRLX2 (400 μg/kg/day/14 days) or vehicle (controls). The collagen:tissue ratio was significantly greater in the aged vehicle treated versus adult bladders (0.57±0.09, p<0.05), while there was no significant difference in vehicle and relaxin treated adult rat bladders (0.41±0.09 versus 0.33±0.09, respectively). In aged bladders, relaxin treatment reversed fibrosis and decreased the collagen:tissue ratio to the level of adult bladders (0.36±0.08, p<0.05).

FIG. 10A-10D. Relaxin reverses age-related bladder fibrosis and treats underactive bladder. Length-tension studies demonstrated that aged male rats had significantly increased passive tension compared to younger males (FIG. 10A), suggesting that there is decreased bladder elasticity and compliance. Decreased compliance was not observed in aged females (not shown). Relaxin treatment (400 μg/kg/day/14 days) of aged males showed a return of passive tension profiles comparable to adults. Histological data correlated with tissue studies, where aged males showed increased bladder collagen content which was decreased following relaxin treatment (FIG. 10B and FIG. 10C). Relaxin also increased expression of L-type Ca²⁺ channels (Cav1.2) in the detrusor layer (FIG. 10D).

FIG. 11. Rat bladders express relaxin receptors. Immunohistochemical and 3,3′-diaminobenzidine (DAB) staining of aged male rat bladders for RXFP1 and RXFP2 (the receptors for hRLX2) showed that RXFP2 is the dominant receptor subtype expressed.

FIG. 12. Relaxin treatment increased detrusor contractility and tissue compliance in aged male, but not female, rat bladders. Length-tension studies demonstrated that 24 month male rats had significantly increased passive tension compared to 9 month males, suggesting decreased bladder elasticity and compliance. hRLX2 treatment (400 μg/kg/day/14 days) of aged males showed a return of passive tension profiles comparable to adults.

FIG. 13. Decreased tissue compliance was not observed in aged females nor was it significantly altered by hRLX2 treatment. Contrary to results in aged male rats, decreased tissue compliance was not observed in aged females nor was it significantly altered by hRLX2 treatment (400 μg/kg/day/14 days).

FIG. 14. Aging results in increased collagen content which is reduced by hRLX2 therapy. Aged male rats showed increased bladder collagen deposition (intense pink staining) which was decreased by hRLX2 treatment (400 μg/kg/day/14 days) to levels comparable with 9 month animals.

FIG. 15. Relaxin treatment increases detrusor smooth muscle Cav1.2 expression. The expression of Cav1.2 in the detrusor (DT) layer was increased in aged rat bladders following hRLX2 treatment (400 μg/kg/day/14 days). The red boxed regions highlight the increased staining for Cav1.2 in treated rats. Note: background staining of the urothelium (UT) is an artifact due to high endogenous peroxidase activity. LP=lamina propria.

FIG. 16. Urine spot analysis from irradiated mice with and without hRLX2 therapy. Mice were placed in metabolic cages with Whatman paper on the floor for 2 hours between 11 AM and 2 PM without access to food and water. Upon completion, filter papers were collected and photographed under UV light. Spot analysis (table, lower left) demonstrated urine leakage suggestive of incontinence as early as 2 weeks post irradiation with decreased voided volumes. hRLX2 increased voided volumes and decreased the number of spots, restoring normal bladder function.

FIG. 17. Expression levels of hRLX2 receptors, RXFP1 and RXFP2, in mouse bladders. Immunohistochemical analysis of RXFP1/2 in the female C57Bl/6 mouse bladders showed that these receptors are expressed on the detrusor smooth muscle (RXFP1/2—green, smooth muscle actin—red, DAPI nuclear stain—blue), with surprisingly little expression in the lamina propria (LP) and urothelium (UT). The expression of RXFP1 (A) was less robust than RXFP2 (B) in histological sections and Western blot analysis (C).

FIG. 18. Endogenous mouse RLX1 (mRLX1) and exogenous hRLX2 blood levels in mice. The plasma levels of endogenous mRLX1 (mouse homologue of hRLX2) and hRLX2 following subcutaneous infusion was determined using ELISA. The levels of mRLX1 were higher in females than in male mice, suggesting females could be less prone to organ fibrosis than males. Mice treated with hRLX2 (2 weeks via subcutaneous osmotic pump) showed a dose-dependent increase in plasma hRLX2 levels where 400 μg/kg/day resulted in a mean level of 17.5 ng/ml.

FIG. 19. Anatomical differences in human and mouse/rat prostates. In humans and other primates, the transitional region of the prostate, where hyperplasia develops, surrounds the urethra and is encapsulated. Accordingly, when BPH develops, it constricts the urethra resulting in increased outlet resistance with a compensatory hypertrophy of the detrusor smooth muscle. In rodents, the lateral and ventral lobes of the prostate can become hyperplastic. While this does not cause outlet obstruction or bladder hypertrophy, it can be detected in histological sections from aged animals. Accordingly, rodents can be useful models for testing hRLX2 in treating BPH.

FIG. 20. Injection sites for selective hRLX2 therapy for benign prostatic hyperplasia (BPH)—in rodents. Different lobes of the rodent prostate (left) can be selectively injected to deliver hRLX2 to regions as shown by selective injection of the ventral lobes of a mouse prostate depicted in the photo image (right).

FIG. 21. Histological sections of male rat urethras—at the level of the prostate—from control and hRLX2 treated aged rats. A. Hyperplasia of the prostate in aged rodents does not constrict the urethra, but causes inflammation, disruption of the columnar epithelium, collagen infiltration and damage to the surrounding internal urethral sphincter which can be determined histologically. B. hRLX2 therapy prevented these adverse changes.

FIG. 22. Applications and mechanisms for hRLX2 in treating lower urinary tract dysfunctions (LUTD). One of the initial responses to inflammation is urothelial apoptosis, disruption of barrier function and urine infiltration. Concurrently, there is damage to the vascular endothelium leading to ischemia. These processes cause increased collagen deposition, and decreased bladder compliance and force generation. Treatment with hRLX2 reverses fibrosis through inhibition of collagen synthesis and enhancement of its degradation by matrix metalloproteinases (MMP). hRLX2 also enhances contractile function through increased Cav1.2 (that is, L-type Ca²⁺ channel) expression and improved tissue perfusion via nitric oxide (NO.) induced vasodilation. hRLX2 is also anti-inflammatory, inhibiting recurrent damage to the bladder wall. Therefore, hRLX2 treatment may be useful for a number of LUTDs including the following: radiation, chemical and interstitial cystitis, bladder over- and under-activity, urinary tract infections, benign prostatic hyperplasia, prostatitis and the consequences of spinal cord injury and aging.

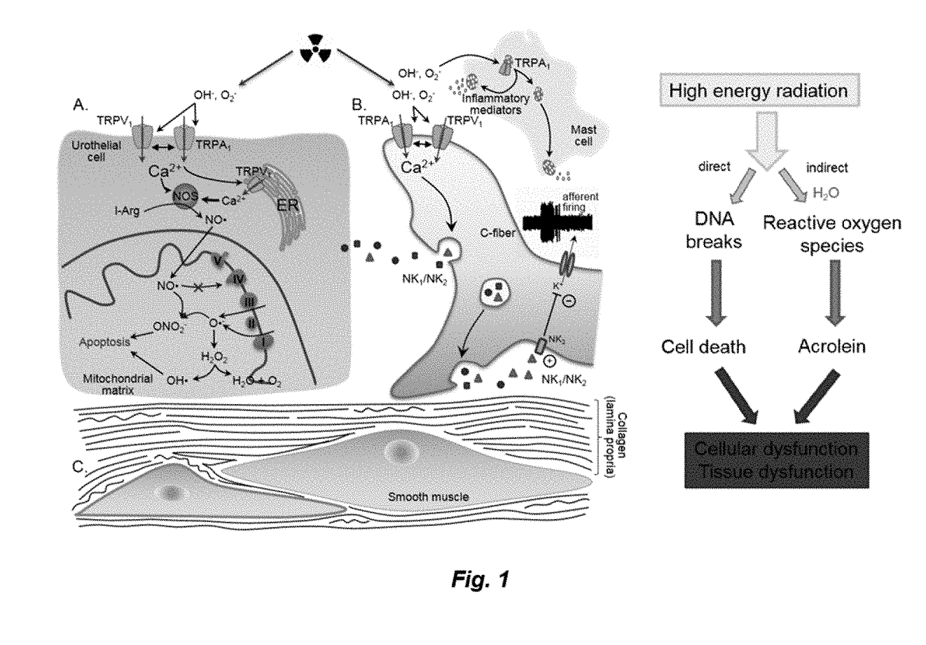

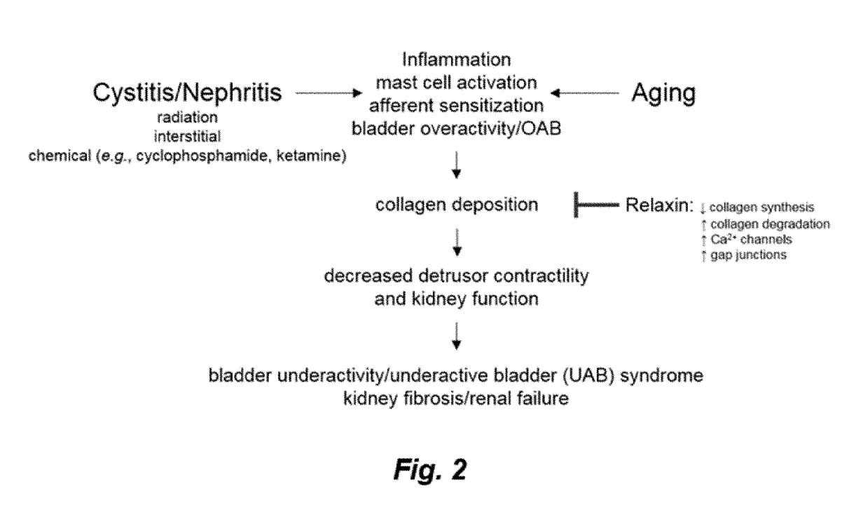

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” a lower urinary tract dysfunction means administration to a patient by any suitable dosage regimen, procedure and/or administration route of a composition, device or structure with the object of achieving a desirable clinical/medical end-point, including but not limited to, stopping, preventing, reversing the development of fibrosis of the bladder, etc.

Provided herein are methods of treating individuals having a lower urinary tract dysfunction with an effective amount of a relaxin peptide, which also can be referred to as an insulin superfamily peptide. A relaxin peptide is a member of the insulin superfamily peptides, including but are not limited to, relaxin-1, relaxin-2, or human insulin-like peptide-3. Additionally, as described herein, a patient in need of a pharmaceutical composition comprising a relaxin peptide is an individual suffering from a lower urinary tract dysfunction. In one aspect, a lower urinary tract dysfunction, is a fibrosis of the bladder. Non-limiting examples of conditions that result in fibrosis of the bladder include benign prostate hyperplasia, radiation or chemical (e.g., cyclophosphamide and ketamine) induced cystitis/nephritis, or age-associated fibrosis of the bladder. By fibrosis, it is meant that there is a thickening and scarring of connective tissue. This may be a consequence of increased collage deposition and/or decreased collagen degradation, a decrease in calcium channels, and a decrease in gap junction expression.

By benign prostate hyperplasia (BPH), it is understood that there is an enlargement of the prostate. The prostate goes through two main growth periods as a man ages. The first occurs early in puberty, when the prostate doubles in size. The second phase of growth begins around age 25 and continues during most of a man's life. As a man ages, his prostate may get larger. Benign prostatic hyperplasia often occurs with the second growth phase. As the prostate enlarges, it can squeeze down on the urethra, causing the bladder wall to become thicker, eventually leading to fibrosis. Eventually, the bladder may weaken and lose the ability to empty completely, leaving some urine in the bladder. The narrowing of the urethra and urinary retention—the inability to empty the bladder completely—cause many of the problems associated with benign prostatic hyperplasia. BPH is common in aging men. About half of all men between the ages of 51 and 60 have BPH. Up to 90% of men over age 80 have BPH.

Radiation cystitis is a complication of radiation therapy to pelvic tumors. The urinary bladder can be irradiated intentionally for the treatment of bladder cancer or incidentally for the treatment of other pelvic malignancies. Manifestations of radiation cystitis can range from minor, temporary, irritative voiding symptoms and painless, microscopic hematuria to more severe complications, such as gross hematuria; contracted, nonfunctional bladder; persistent incontinence; fistula formation; necrosis; and death. Tumors of the pelvic organs (for example, prostate, bladder, colon, rectum) are common in men, constituting 35% of new cancer diagnoses for 2017. In women, cancer of the colon and rectum, bladder, and genital tract (uterus, ovary, and vagina/vulva) constitute 18% of new cancer diagnoses in 2017. Radiation therapy is an important management tool for the treatment of these malignancies, creating significant potential for the development of irradiation injury to the bladder. There are three phases of radiation cystitis. An acute inflammatory response with tissue edema and hyperemia develops within 4-6 weeks. This is much later compared to the intestinal epithelium, since the regeneration time of the urothelium is slower. The acute reaction is followed either by healing or is proceeded by the second (ischemic) phase of tissue reaction. The second phase of the radiation injury leads to an ischemic tissue damage due to necrosis of the vascular endothelium and perivascular fibrosis. Histology reveals the picture of obliterative endarteritis. The ischemic bladder wall becomes more sensitive to external factors like bacterial infection and the ability to heal is reduced significantly. Symptoms of the ischemia are recurrent hematuria and there is an increased risk for bladder fistulas. In the course of recurrent ischemia, the bladder walls react with progressive fibrosis and shrinkage. The fibrotic shrinkage may occur up to 10 years after radiation therapy. Some signs and symptoms of radiation cystitis include dysuria, frequency, nocturia, recurrent hematuria, recurrent urinary tract infections, signs of the bladder fistula are persisting urinary tract infections, urinary incontinence and pneumaturia.

Fibrosis may also result from the use of various drugs/chemicals. Certain medications, such as cyclophosphamide and ketamine, can cause inflammation of the bladder as the broken-down components of the drugs exit the body. This injury to the bladder ultimately leads to the thickening and scarring of the bladder wall tissue.

Additionally, collagen deposition and decreased bladder compliance can occur with aging leading to bladder overactivity or underactivity/UAB syndrome. Overactivity commonly degrades to underactivity. By overactivity it is meant individuals suffer sudden, and uncontrollable urges to urinate. Some people will leak urine when they feel the urge (“incontinence”). Having to go to the bathroom many times during the day and night is another symptom of overactive bladder. Causes of overactive bladder include, but are not limited to bacterial infections, tumors, bladder stones, and neurogenic or myogenic etiologies.

By underactivity it is meant that patients with an underactive bladder can hold unusually large amounts of urine, but have a diminished sense of when the bladder is full and are not able to contract the detrusor sufficiently, resulting in incomplete bladder emptying. The underactive bladder is a symptom complex suggestive of detrusor underactivity and is usually characterized by prolonged urination time with or without a sensation of incomplete bladder emptying, usually with hesitancy, reduced sensation on filling, and a slow stream. Detrusor underactivity is a medical diagnostic term based on urodynamic testing that requires catheter insertion into the bladder and rectum. Causes of underactive bladder include, but are not limited to, neurogenic, myogenic, aging, and medication side effects. Underactive bladder may be caused by a maladaptation. For instance, neurological disorders or loss of neurological signaling as a result of aging.

Interstitial cystitis or painful bladder syndrome/bladder pain syndrome is a chronic inflammatory condition of unknown etiology which is characterized by mild discomfort to severe pelvic pain during bladder filling. This condition can result in decreased bladder capacity and urinary frequency. Although the underlying cause of interstitial cystitis is unclear, it is commonly associated with increased urothelial permeability and/or damage (e.g., Hunner's ulcers). This can cause urine infiltration and further potentiate inflammation that can progress to bladder wall fibrosis.

Patients with spinal cord injury are likely to experience loss of bladder sensation and the ability to voluntarily void which can be accompanied with detrusor sphincter dyssynergia causing bladder outlet obstruction and vesico-ureteral reflux. As such, patients may need to perform intermittent catheterization to allow complete emptying of their bladders to prevent renal damage. The multiple, daily catheterizations can lead to frequent urinary tract infections and chronic inflammation which contribute to bladder fibrosis and detrusor smooth muscle decompensation.

Prostatitis can be induced by bacterial infections or be idiopathic in nature (i.e., non-bacterial). The non-bacterial form of prostatitis can be associated with poorly controlled recurrent inflammation that can promote collagen deposition and prostatic hyperplasia. This in turn, could increase bladder outlet resistance to further exacerbate inflammation and fibrosis in the urinary bladder.

Diabetes mellitus type-2 (insulin-resistant) has been demonstrated to promote inflammation within the bladder wall, which chronically leads to detrusor hypocontractility and bladder wall fibrosis (Golbidi S. et al.: Bladder Dysfunction in Diabetes Mellitus, Front Pharmacol, 1: 136, 2010).

Recurrent urinary tract infections (UTIs) and/or chronic bacterial cystitis can also be underlying causes for bladder inflammation and fibrosis. The risk of recurrent urinary tract infections and/or chronic bacterial cystitis is significantly increased by catheterization or diabetes mellitus (Nitzan O., et al.: Urinary tract infections in patients with type 2 diabetes mellitus: review of prevalence, diagnosis, and management. Diabetes Metab Syndr Obes.; 8:129-36, 2015).

A relaxin peptide (generally, “a relaxin”) is a member of the relaxin family of peptide hormones including, in humans, relaxin-1, relaxin-2, relaxin-3, insulin-like peptide 3 (INSL3), insulin-like peptide 4 (INSL4), insulin-like peptide 5 (INSL5), and insulin-like peptide 6 (INSL6). As exemplary of the general structure and processing of relaxins as a class, mature human relaxin 2 is a hormonal peptide of approximately 6000 daltons in molecular weight. Naturally occurring relaxin is synthesized as a single-chain 23 kDa preprorelaxin with the overall structure: signal peptide, B-chain, connecting C-peptide, and A-chain. During the biosynthesis of relaxin, the signal peptide is removed as the nascent chain is moved across the endoplasmic reticulum producing the 19-kDa prorelaxin. Further processing of the prorelaxin to relaxin occurs in vivo through the endoproteolytic cleavage of the C-peptide at specific pairs of basic amino acid residues located at the B/C-chain and A-/C-chain junctions after the formation of disulfide bridges between the B- and A-chains in a manner analogous to insulin. The relaxin disulfide bridges occur between the cysteines at A9-B10 and A22-B22 with an intra-chain disulfide bridge within the A-chain between A8 and A13. Relaxins are found in other vertebrate species, with many well-characterized variants that are potentially useful interspecies, including mammalian relaxins.

The amino acid sequences of relaxins have been determined by direct protein sequencing or deduced from the nucleotide sequences of the DNAs for a number of species.

An exemplary sequence for human relaxin-2 is provided below (GENBANK Accession No. AAI26416) (see also, Strausberg, R. L., et al.: Generation and initial analysis of more than 15,000 full-length human and mouse cDNA sequences, Proc. Natl. Acad. Sci. U.S.A. 99 (26), 16899-16903 (2002)):

(SEQ ID NO: 1)
MPRLFFFHLL GVCLLLNQFS RAVADSWMEE VIKLCGRELV
RAQIAICGMS TWSKRSLSQE DAPQTPRPVA EIVPSFINKD
TETINMMSEF VANLPQELKL TLSEMQPALP QLQQHVPVLK
DSSLLFEEFK KLIRNRQSEA ADSSPSELKY LGLDTHSRKK
RQLYSALANK CCHVGCTKRS LARFC

An exemplary sequence for relaxin-1 (NCBI Gene ID: 6013, OMIM (Online Mendelian Inheritance in Man): 179730, NCBI Reference Sequence: NP_008842.1 (prorelaxin H1 preproprotein [Homo sapiens]),) is:

(SEQ ID NO: 2)
MPRLFLFHLL EFCLLLNQFS RAVAAKWKDD VIKLCGRELV
RAQIAICGMS TWSKRSLSQED APQTPRPVAE IVPSFINKDT
ETIIIMLEFI ANLPPELKAA LSERQPSLPE LQQYVPALKD
SNLSFEEFKK LIRNRQSEA ADSNPSELKY LGLDTHSQKK
RRPYVALFEK CCLIGCTKRS LAKYC

Insulin-like peptide 3 (INSL3) is a member of the insulin-relaxin family of structurally related peptides, which has evolved by sequential duplication from a common ancestor. The peptide is processed similarly to the other relaxin peptides, as described above. INSL3 is expressed in large amount by fetal and adult-type Leydig cells, once these have attained a mature phenotype. INSL3 acts through a G protein-coupled receptor called RXFP2 (relaxin family peptide receptor 2; previously called LGR8), which appears to be expressed in multiple tissues, including germ cells, where INSL3 seems to act as a survival or antiapoptotic factor. However, the principal function for INSL3, is in the male fetus, during the first phase of testicular descent, where INSL3 from the fetal Leydig cells promotes the growth and expansion of the gubernaculum, retaining the embryonic testis in the inguinal region.

An exemplary sequence for human insulin-like peptide-3 is provided below (GENBANK Accession No. NP_001252516) (see also, Huang X, et al.: Mutational screening of the INSL3 gene in azoospermic males with a history of cryptorchidism Andrologia 48 (7), 835-839 (2016)):

(SEQ ID NO: 3)
MDPRLPAWAL VLLGPALVFA LGPAPTPEMR EKLCGHHFVR
ALVRVCGGPR WSTEARRPAT GGDQRESHSV SQAGLKLLSS
SNPPTLTFQS VGISDVSCYS GWRDDICSMG WWPTVISRWD
LACSPCPRPL TITATTVQLP PTLHATAASV AVPNKTC

SERELAXIN (RLX030) is a recombinant form of human relaxin-2 that is currently being tested for the treatment of acute heart failure (AHF). The peptide sequence for Serelaxin is the same as native RLX2.

Unless otherwise specified, “relaxin” or “relaxin peptide”, refers collectively to the RLX1, RLX2, RLX3, INSL3, INSL4, INSL5, and INSL6 proteins, and alleles and polymorphisms thereof. “relaxin-2”, or “relaxin-2 peptide” refers to without limitation, a peptide hormone that is identical to relaxin-2 in its amino acid sequence, and includes “Serelaxin”. “relaxin-2” encompasses human relaxin-2, including intact full-length human relaxin-2 or a portion of the relaxin-2 molecule that retains biological activity. “relaxin-2” encompasses human H1 preprorelaxin-2, prorelaxin-2, and relaxin-2; H2 preprorelaxin-2, prorelaxin-2, and relaxin-2; and H3 preprorelaxin-2, prorelaxin-2, and relaxin-2. “relaxin-2” further includes biologically active (“pharmaceutically active”) relaxin-2 from recombinant, synthetic or native sources as well as relaxin-2 variants, such as amino acid sequence variants. As such, “relaxin-2” includes synthetic human relaxin-2 and recombinant human relaxin-2, including synthetic H1, H2 and H3 human relaxin-2 and recombinant H1, H2 and H3 human relaxin-2. “relaxin-2 analogs” include active agents with relaxin-2-like activity, such as relaxin-2 agonists, “relaxin-2” derivatives include synthetically-modified relaxin-2 sequences and portions thereof that retain biological activity, including all agents that competitively displace bound relaxin-2 from a relaxin-2 receptor (e.g., RXFP1 receptor, RXFP2 receptor, RXFP3 receptor, RXFP4 receptor, previously known as LGR7, LGR8, GPCR135, GPCR142, respectively). Thus, a pharmaceutically effective relaxin-2 or relaxin-2 analog or derivative is any agent with relaxin-2-like activity that is capable of binding to a relaxin-2 receptor to elicit a relaxin-2-like response. In addition, a pharmaceutically effective relaxin-2 or relaxin-2 analog or derivative is any agent with relaxin-2-like activity that is capable of up-regulating and/or modifying the sST-2 decoy receptor activity, and further capable of down-regulating and/or modifying the IL-33 activity, thereby modulating and/or changing and/or decreasing the amount of proinflammatory cytokines that are present in a tissue and/or organ during and/or at the onset of inflammation. In addition, the nucleic acid sequence of a relaxin-2 derivative as used herein must not be 100% identical to nucleic acid sequence of human relaxin-2 (e.g., H1, H2 and/or H3) but may be at least about 40%, 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the nucleic acid sequence of human relaxin-2. relaxin-2 and relaxin-2 analogs or derivatives, as used herein, can be made by any method known to those skilled in the art. Examples of such methods are illustrated, for example, in U.S. Pat. No. 5,759,807, incorporated herein by reference for its technical disclosure. Examples of relaxin-2 molecules and analogs are illustrated, for example, in U.S. Pat. No. 5,166,191, incorporated herein by reference for its technical disclosure. Biologically active relaxin-2 may be derived from human, murine (e.g., rat or mouse), porcine, or other mammalian sources.

In calculating percent sequence identity, two sequences are aligned and the number of identical matches of amino acid residues between the two sequences is determined. The number of identical matches is divided by the length of the aligned region (i.e., the number of aligned amino acid residues) and multiplied by 100 to arrive at a percent sequence identity value. The length of the aligned region can be a portion of one or both sequences up to the full-length size of the shortest sequence. Alignment of two or more sequences to determine percent sequence identity can be performed using the computer program ClustalW2 (EMBL-EBI) and default parameters, which calculates the best match between a query and one or more subject sequences, and aligns them.

Relaxin-2-containing agents may be modified to increase in vivo half-life, e.g., PEGylated relaxin-2 (e.g., a relaxin-2 conjugated to a polyethylene glycol), modifications of amino acids in relaxin-2 that are subject to cleavage by degrading enzymes, and the like. “Relaxin-2” also includes relaxin-2 A and B chains having N- and/or C-terminal truncations. In general, for H2 relaxin-2, the A chain can be varied from A(1-24) to A(10-24) and B chain from B(1-33) to B(10-22); and in H1 relaxin-2, the A chain can be varied from A(1-24) to A(10-24) and B chain from B(1-32) to B(10-22). Also included within the scope of the term “relaxin-2” or “relaxin-2 derivatives” are other insertions, substitutions, or deletions of one or more amino acid residues, glycosylation variants, unglycosylated relaxin-2, organic and inorganic salts, covalently modified derivatives of relaxin-2, preprorelaxin-2, and prorelaxin-2. Also encompassed in the term is a relaxin-2 derivative having an amino acid sequence, which differs from a wild-type (e.g., naturally-occurring) sequence, including, but not limited to, relaxin-2 analogs disclosed in U.S. Pat. No. 5,811,395, incorporated herein by reference for its technical disclosure. Possible modifications to relaxin-2 amino acid residues include the acetylation, formylation or similar protection of free amino groups, including the N-terminal, amidation of C-terminal groups, or the formation of esters of hydroxyl or carboxylic groups, e.g., modification of the tryptophan (Trp) residue at B2 by addition of a formyl group. The formyl group is a typical example of a readily-removable protecting group. Other possible modifications include replacement of one or more of the natural amino-acids in the B and/or A chains with a different amino acid (including the D-form of a natural amino-acid), including, but not limited to, replacement of the Met moiety at B24 with norleucine (Nle), valine (Val), alanine (Ala), glycine (Gly), serine (Ser), or homoserine (HomoSer). Other possible modifications include the deletion of a natural amino acid from the chain or the addition of one or more extra amino acids to the chain.

Additional modifications include amino acid substitutions at the B/C and C/A junctions of prorelaxin-2, which modifications facilitate cleavage of the C chain from prorelaxin-2; and variant relaxin-2 comprising a non-naturally occurring C peptide, e.g., as described in U.S. Pat. No. 5,759,807, incorporated herein by reference for its technical disclosure. “Relaxin-2” also includes fusion polypeptides comprising relaxin-2 and a heterologous polypeptide. A heterologous polypeptide (e.g., a non-relaxin-2 polypeptide) fusion partner may be C-terminal or N-terminal to the relaxin-2 portion of the fusion protein. Heterologous polypeptides include immunologically detectable polypeptides (e.g., comprising “epitope tags”); polypeptides capable of generating a detectable signal (e.g., comprising green fluorescent protein, an enzyme such as alkaline phosphatase, and others known in the art); therapeutic polypeptides, including, but not limited to, cytokines, chemokines, and growth factors. All such variations or alterations in the structure of the relaxin-2 molecule resulting in variants are included within the scope of this disclosure so long as the functional (biological) activity of the relaxin-2 is maintained. Any modification of the relaxin-2 amino acid sequence or structure is typically one that does not increase its immunogenicity in the individual being treated with the relaxin-2 variant. Those variants of relaxin-2 having the described functional activity can be readily identified using in vitro and in vivo assays known in the art.

Derivatives, variants, or analogs of relaxins, including but not limited to human relaxin 1, human relaxin 2, and human insulin-like peptide 3 include modified peptides as described above in the context of relaxin 2, to the extent applicable to each specific relaxin, and includes modified polypeptides having substantial sequence identity with the particular relaxin, so that functionality of the relaxin derivative, variant, or analog is substantially retained in the context of the methods described herein.

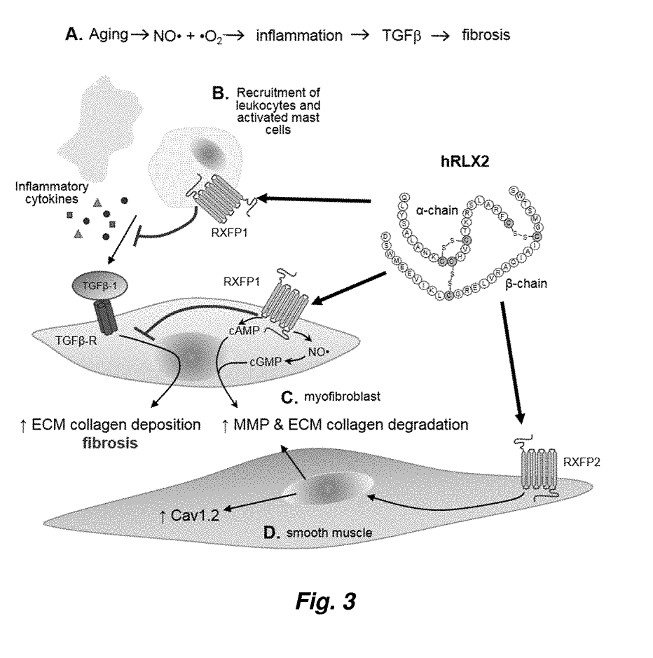

The mechanisms of action of relaxins are briefly described as follows. The actions of relaxins are mediated via its receptors, RXFP1/2 which are G-Protein Coupled Receptors (GPCR) that produce transient elevations of cAMP and activation of downstream kinases. In spontaneously hypertensive and in aged rats, human relaxin-2 treatment for 2 weeks has been found to modulate Wnt signaling by pathways that are not yet fully understood. Canonical Wnt signaling is believed to stimulate members of the frizzled receptor family to initiate the translocation of β-catenin from the cell membrane to the nucleus to initiate collagen deposition, remodeling and fibrosis (Bastakoty, D., et al.: Wnt/beta-catenin pathway in tissue injury: roles in pathology and therapeutic opportunities for regeneration. FASEB J, 2016) which can be opposed by relaxin-2 binding to one of its four receptors. Relaxin-2 binding is believed to increase PKA which inhibits β-catenin but may also stimulate a pathway leading to gene transcription and increased ECM metalloproteinases (Ahmad, N., et al.: Relaxin induces matrix-metalloproteinases-9 and -13 via RXFP1: induction of MMP-9 involves the PI3K, ERK, Akt and PKC-zeta pathways. Mol Cell Endocrinol, 363: 46, 2012) and voltage-gated Ca²⁺ channel current (Han, X., et al.: Relaxin increases heart rate by modulating calcium current in cardiac pacemaker cells. Circ Res, 74: 537, 1994) along with decreased collagen synthesis (Samuel, C. S.: Relaxin: antifibrotic properties and effects in models of disease. Clin Med Res, 3: 241, 2005). A theoretical mechanism for relaxin's actions is depicted in FIG. 3.

Relaxin has been described as being effective for cardiovascular therapy (see for instance, U.S. Pat. No. 5,166,191, incorporated herein by reference for its technical disclosure), in the treatment of fibromyalgia (see for instance, U.S. Pat. No. 5,707,642, incorporated herein by reference for its technical disclosure), and in the treatment of multiple sclerosis (see for instance, United States Patent Application Publication No. 2014/0256633, incorporated herein by reference for its technical disclosure). Additionally, relaxin has undergone phase I clinical trials for treating acute heart failure (Teerlink, J. R., et al.: Serelaxin, recombinant human relaxin-2, for treatment of acute heart failure (RELAX-AHF): a randomised, placebo-controlled trial. Lancet, 381: 29, 2013) and scleroderma (Seibold, J. R., et al.: Recombinant human relaxin in the treatment of scleroderma. A randomized, double-blind, placebo-controlled trial. Ann Intern Med, 132: 871, 2000) and both studies have validated its safety for use in humans at concentrations as high as 250 μg/kg/day.

Compositions containing a relaxin peptide, as described herein, may be adapted for administration by any appropriate route of administration. For instance, suitable routes of administration include, but are not limited to oral (including buccal or sublingual), nasal, topical (including buccal, sublingual or transdermal) or parenteral (including subcutaneous, intramuscular, intravenous or intradermal) route. As will be appreciated by the skilled artisan, the route and/or mode of administration will vary depending upon the desired results.

Compositions containing a relaxin peptide may be prepared by any method known in the art of pharmacy, for example by bringing into association the active ingredient with the carrier(s) or excipient(s). As used herein, “carrier” or “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it may be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the insulin superfamily peptide. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

Additionally, compositions containing a relaxin peptide may be in variety of forms. The preferred form depends on the intended mode of administration and therapeutic application, which will in turn dictate the types of carriers/excipients. Suitable forms include, but are not limited to, liquid, semi-solid and solid dosage forms.

Pharmaceutical formulations adapted for oral administration may be presented, for example and without limitation, as discrete units such as capsules or tablets; powders or granules; solutions or suspensions in aqueous or non-aqueous liquids; edible foams or whips; or oil-in-water liquid emulsions or water-in-oil liquid emulsions. In certain embodiments, an insulin superfamily peptide may be contained in a formulation such that it is suitable for oral administration, for example, by combining the relaxin peptide with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Pharmaceutical formulations adapted for transdermal administration may be presented, for example and without limitation, as discrete patches intended to remain in intimate contact with the epidermis of the recipient for a prolonged period of time or electrodes for iontophoretic delivery.

Pharmaceutical formulations adapted for topical administration may be formulated, for example and without limitation, as ointments, creams, suspensions, lotions, powders, solutions, pastes, gels, sprays, aerosols or oils.

Pharmaceutical formulations adapted for nasal administration wherein the carrier is a solid include a coarse powder having a particle size for example in the range 20 to 500 microns which is administered in the manner in which snuff is taken, that is, by rapid inhalation through the nasal passage from a container of the powder held close up to the nose. Suitable formulations wherein the carrier is a liquid, for administration as a nasal spray or as nasal drops, include aqueous or oil solutions of the active ingredient. Pharmaceutical formulations adapted for administration by inhalation include, without limitation, fine particle dusts or mists which may be generated by means of various types of metered dose pressurized aerosols, nebulizers or insufflators.

Pharmaceutical formulations adapted for parenteral administration include aqueous and non-aqueous sterile injection solutions which may contain, for example and without limitation, anti-oxidants, buffers, bacteriostats, lipids, liposomes, emulsifiers, also suspending agents and rheology modifiers. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Therapeutic compositions typically must be sterile and stable under the conditions of manufacture and storage. For example, sterile injectable solutions can be prepared by incorporating the active compound (that is, a relaxin) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The pharmaceutical compositions of the invention include a “therapeutically effective amount” or a “prophylactically effective amount” of a relaxin. For instance, a pharmaceutical composition comprising human relaxin-1, human relaxin-2, or human insulin-like peptide-3. A “therapeutically effective amount” refers to an amount effective, at dosages and for periods of time necessary, to achieve the desired therapeutic result. An “amount effective” for treatment of a condition is an amount of an active agent or dosage form, such as the coacervate composition described herein, effective to achieve a determinable end-point. The “amount effective” is preferably safe—at least to the extent the benefits of treatment outweighs the detriments and/or the detriments are acceptable to one of ordinary skill and/or to an appropriate regulatory agency, such as the U.S. Food and Drug Administration. A therapeutically effective amount of a relaxin peptide may vary according to factors such as the disease state, age, sex, and weight of the individual, and the ability of the insulin superfamily peptide to elicit a desired response in the individual. A therapeutically effective amount is also one in which any toxic or detrimental effects of the relaxin peptide are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, ever 10, 15, 20, 30, 45, 60, 90, or 120 minutes, every 2 through 12 hours daily, or every other day, etc. be proportionally reduced or increased as indicated by the exigencies of the therapeutic situation. In some instances, it may be especially advantageous to formulate parenteral compositions in dosage unit form for ease of administration and uniformity of dosage. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting, dosing regimen may comprise treating a patient in need thereof with 1-500 μg/kg/day, 1-200 μg/kg/day, or 30 μg/kg/day of a pharmaceutical composition comprising a relaxin, such as a human relaxin-1, a human relaxin-2, or a human insulin-like peptide-3, intravenously in order to obtain a circulating level of 1-70 ng/mL in the blood stream. An alternative delivery of a pharmaceutical composition comprising a relaxin, such as a human relaxin-1, a human relaxin-2, or a human insulin-like peptide-3, would be subcutaneously by a mini-pump of 1-400 μg/kg/day for 2 weeks in order to yield a level of 1-70 ng/mL in the plasma. The ultimate dose will of course depend on the route of administration and the age, weight and condition of the patient and will be at the doctor's discretion.

In the context of lower urinary tract dysfunctions, it may be useful to administer a pharmaceutical composition comprising a relaxin peptide directly to the bladder. For instance, the pharmaceutical composition comprising, for example and without limitation, from 1-400 μg/kg/day of relaxin-2 peptide, may be administered to a patient in need via sterile technique catheterization.

The following examples are provided for illustration purposes.

### Example 1: Relaxin Reverses Radiation Cystitis Injuries

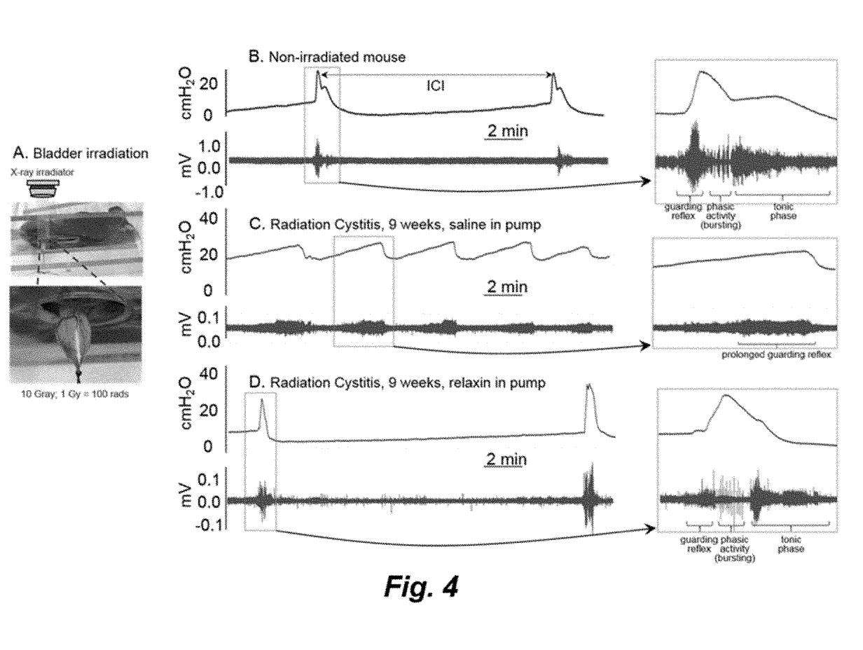

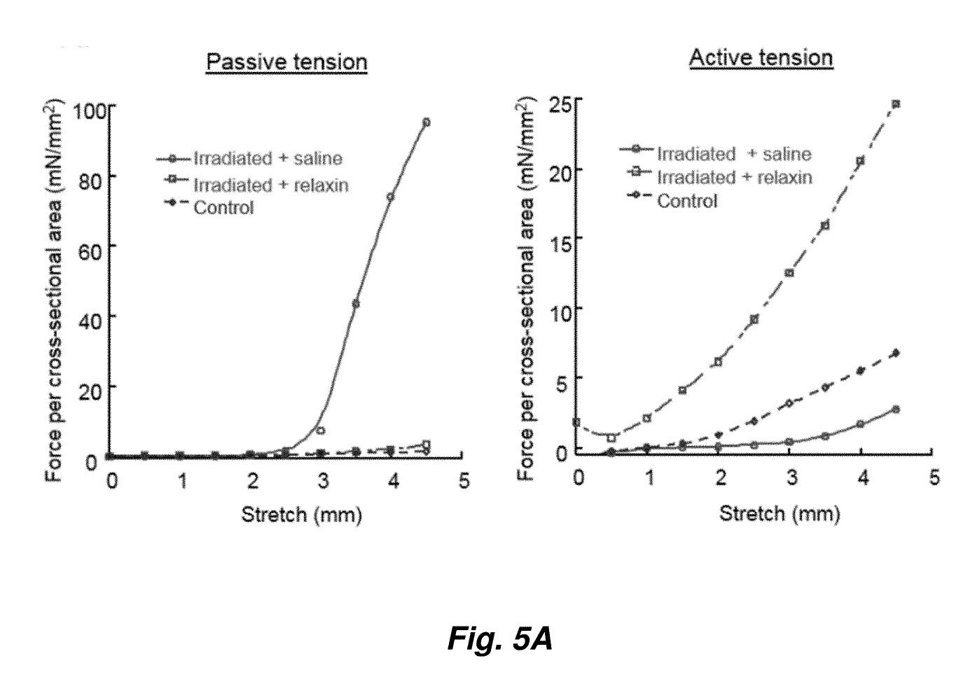

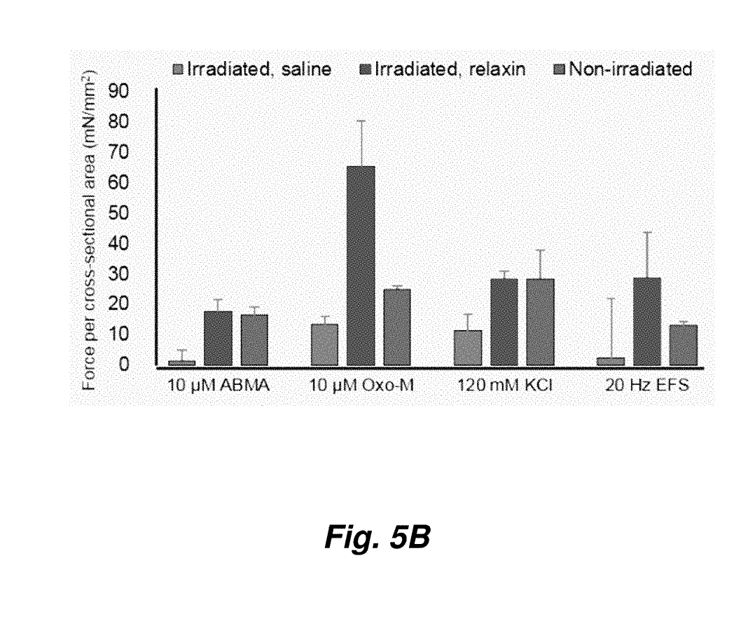

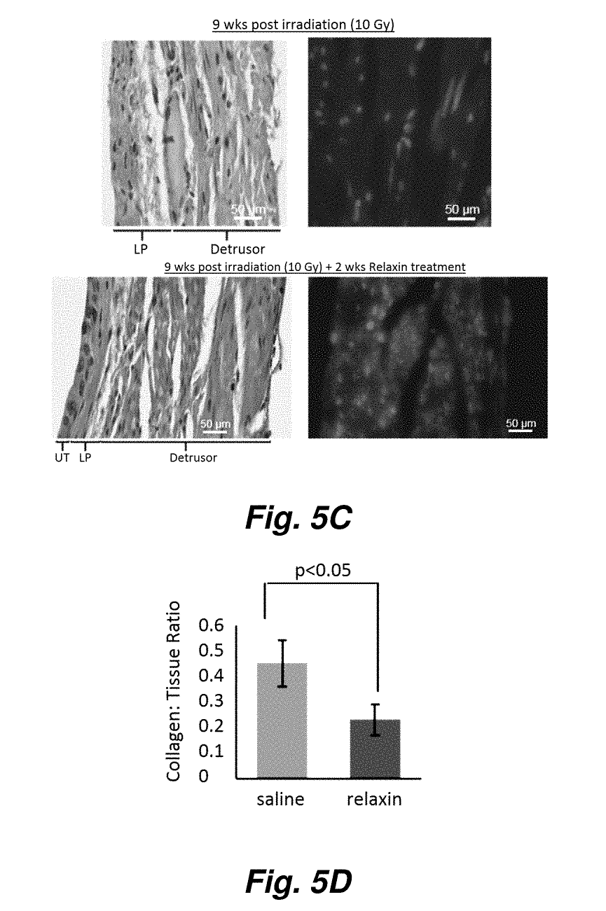

A mouse model was developed to mimic chronic radiation cystitis. As a surrogate to chronic radiation cystitis in the mouse, a laparotomy was performed where the bladder was briefly withdrawn for selective high dose (10 Gy) irradiation (FIG. 4(A)). Delivered to the pelvic region such a dose could be lethal (LD₅₀≅8 Gy). By 9 weeks following selective bladder irradiation, cystometry revealed that animals were unable to void and exhibited overflow incontinence as shown in FIG. 4(C). We attribute this inability of the bladder to empty normally to decreased compliance due to chronic fibrosis (as demonstrated in FIGS. 5A-5D). The corresponding external urethral sphincter (EUS) electromyogram (green traces) demonstrates that the animals had prolonged guarding reflexes and that bursting did not occur. However, when relaxin was administered for 2 weeks starting at week 7 post-irradiation (400 μg/kg/day infused subcutaneously using implantable ALZET mini-pumps), the cystometrograms and electromyograms (FIG. 4(D)) were similar to those seen in non-irradiated mice (FIG. 4(B)) with the return of a normal guarding reflex and bursting (FIG. 4(D), right panel) which permits the animals to void. It is important to note that while human and rodent sphincters exhibit a guarding reflex as bladder pressures approach threshold, the sphincter in humans completely relaxes to permit voiding whereas in rodents it undergoes bursting with intermittent phasic activity during which tonic activity decreased permitting voiding to occur (FIG. 4(B) and FIG. 4(D)). Moreover, following spinal cord injury, rodents can develop detrusor-sphincter-dyssynergia (DSD) which also occurs in humans. In length-tension studies, there was a dramatic increase in passive tension in irradiated mouse bladders which was reversed by relaxin and which also increased active force generation (FIG. 5A). This demonstrates an improvement of contractile properties of the muscle with the beneficial effects due to a combination of relaxin-dependent remodeling, namely: (1) an increase in the cholinergic response (FIG. 5B); (2) decreased collagen content (FIG. 5C; left panels and FIG. 5D); and (3) an increase in the expression of Cav1.2α (the primary subunit peptide Cav1.2α1C subunit that encodes for the L-type Ca²⁺ channel current, I_{Ca,L}) as demonstrated using immunohistochemistry (FIG. 5C; right panels). This is the first application of relaxin to treat bladder dysfunction due to irradiation damage or any urinary tract disease. The data also are by far the most impressive reversal/treatment for radiation cystitis.

### Example 2: Bladder Underactivity/Underactive Bladder (UAB) Syndrome

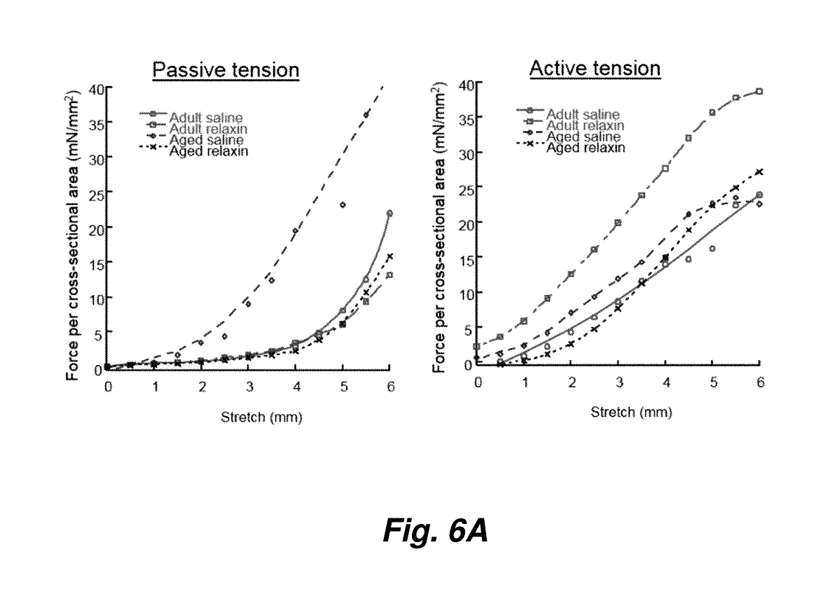

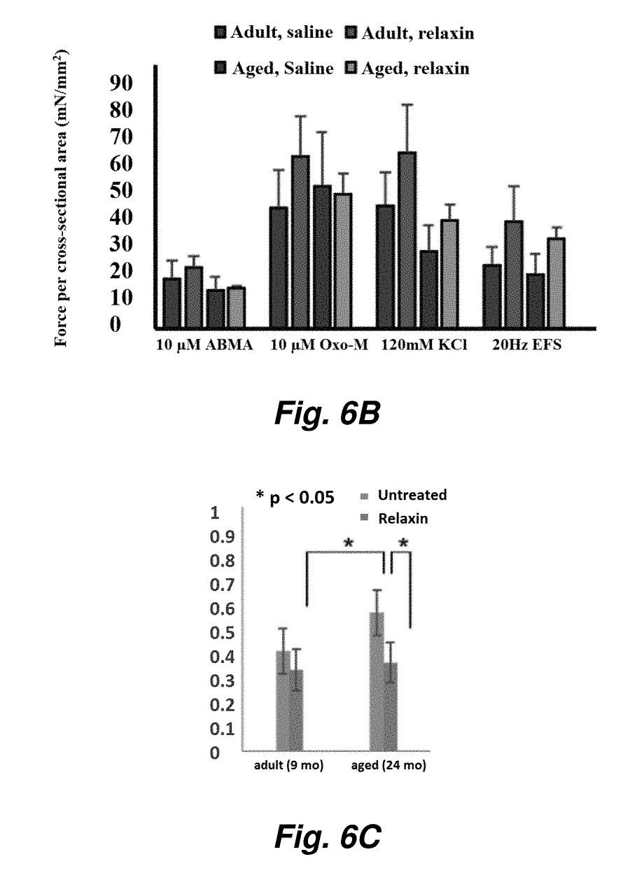

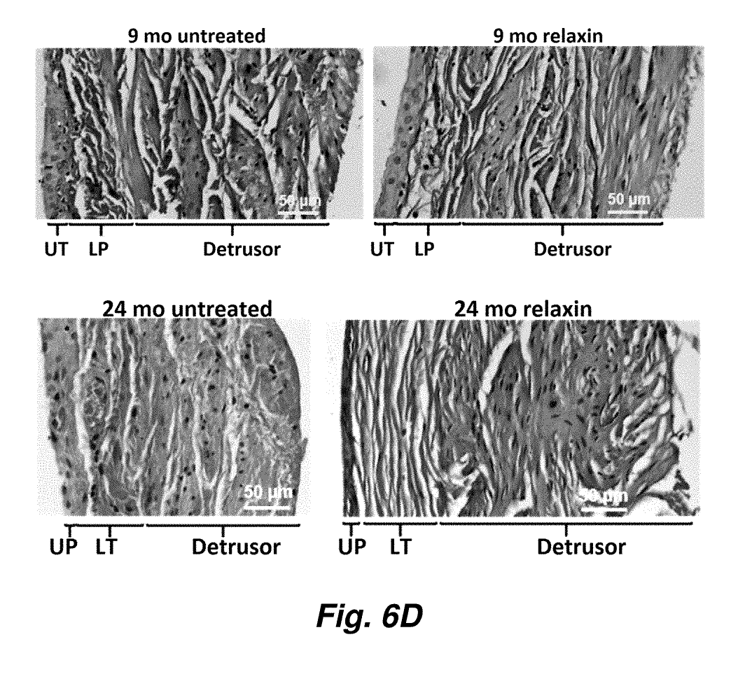

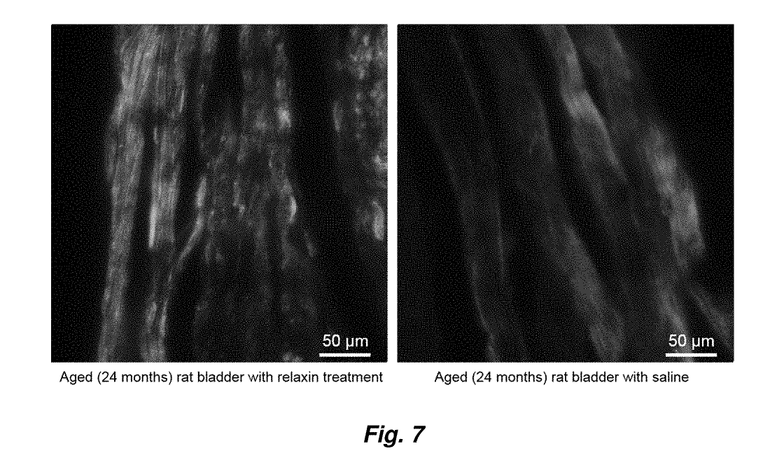

In studies using Fischer rats (F-344) from the NIA, 24 month-old aged animals exhibited substantial increases in passive tension not seen in 9 month-old adults, while active tensions were comparable among the two age groups (FIG. 6A). However, following 2 weeks of treatment with relaxin (400 μg/kg/day infused subcutaneously using ALZET pumps), there were substantial increases in both compliance and force generation in the aged rats. These increases in force generation were not due to enhanced cholinergic or purinergic transmitter release (FIG. 6B), but rather decreases in collagen content (FIG. 6C and FIG. 6D) and increases in smooth muscle expression of Cav1.2α, as demonstrated using immunohistochemistry (FIG. 7). This is the first use of relaxin to treat bladder dysfunction due to aging and may be the first effective treatment for bladder underactivity/UAB syndrome in the elderly.

### Example 3: Relaxin for Treatment of Underactive Bladder

We hypothesize that relaxin may be therapeutic in treating bladder underactivity in the elderly for which there is no effective treatment. Aims of this study were to test the effect of systemically administered relaxin on bladder smooth muscle function in aged versus adult rats.

Study Design, Materials and Methods:

Adult (9 months old) and aged (24 months old) Fisher 344/Brown Norway F1 (F-344) male rats were used in this study. Six out of twelve rats in each age group were treated with relaxin (400 μg/kg/day) or vehicle which were infused by osmotic mini-pumps (ALZET) for 14 days, after which bladders were excised and cut from outlet to dome along the midline ventral and dorsal aspects to form two strips.

One strip was placed in a recording chamber with oxygenated Krebs solution. The base was pinned to a fixed platform and the dome connected to a tension transducer mounted on a programmable stepper motor. Bladder strips were stretched longitudinally in 500 μm increments and baseline tension allowed to stabilize (passive tension). Three field stimulation (20 Hz, 3 sec train, 0.5 ms pulse width, 15V output) contractions were performed at each stretch to determine active force generation.

The other bladder strip was fixed in 10% PFA, embedded in paraffin, cut 5 μm thick, stained for collagen (Sigma HT251) and examined using bright field microscopy (total mag., 400×). The images were obtained at identical conditions and color analysis was performed using the ImageJ software threshold function to discriminate collagen and smooth muscle content.

Results:

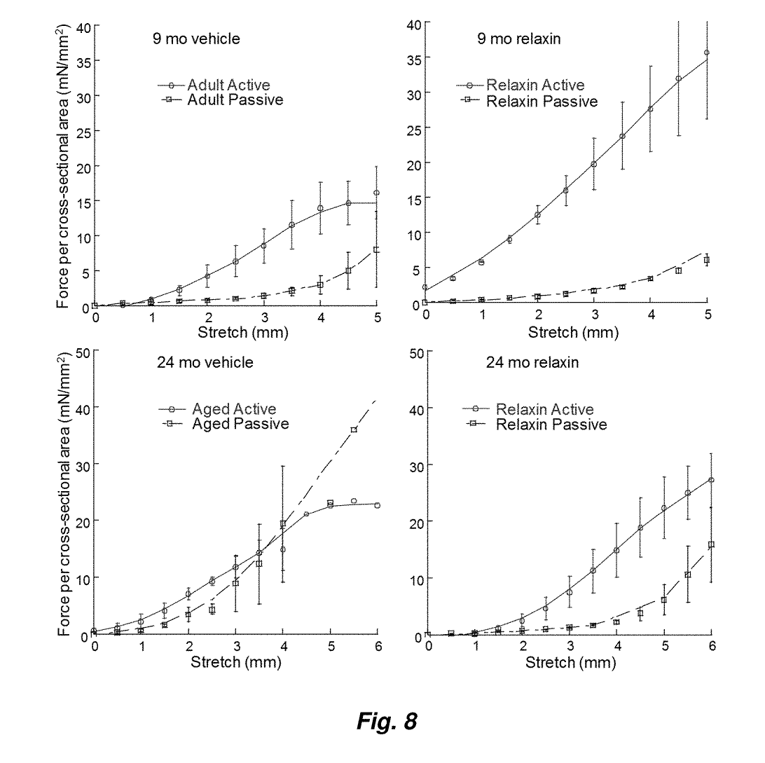

In aged rats, passive tension began increasing at a smaller degree of stretch compared to relaxin treated animals (FIG. 8). Active tension profiles were equivocal between relaxin and vehicle treated aged rat bladders. There was no significant difference in the passive tension profile of relaxin versus vehicle treated adult rat bladders. However, relaxin treated adult rat bladders showed increased active tension generation compared to controls.

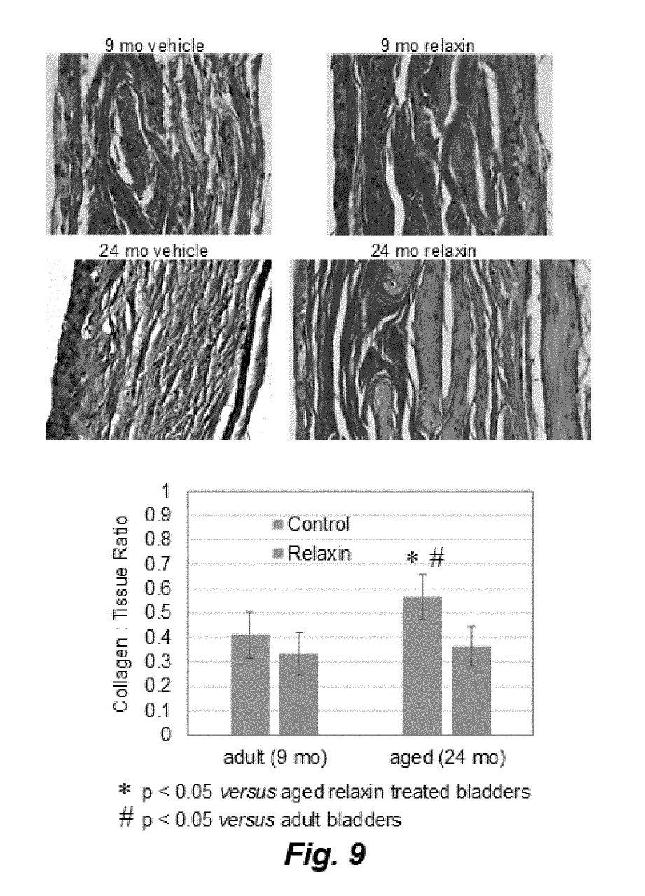

The collagen:tissue ratio was significantly greater in the aged vehicle treated versus adult bladders (0.57±0.09, p<0.05), while there was no significant difference in vehicle and relaxin treated adult rat bladders (0.41±0.09 versus 0.33±0.09, respectively). In aged bladders, relaxin treatment reversed fibrosis and decreased the collagen:tissue ratio to the level of adult bladders (0.36±0.08, p<0.05). Representative histological sections are shown in FIG. 9.

Interpretation of Results:

The leftward shift in the passive length-tension curves in aged vehicle treated rats demonstrates a significant decrease in detrusor compliance due to increased collagen and decreased muscle content which was reversed by relaxin treatment. Relaxin therefore, is expected to be therapeutically beneficial in treating age-related bladder underactivity by decreasing collagen and increasing muscle content thereby improving bladder contractility.

### Example 4: Relaxin Reverses Age-Related Bladder Fibrosis and Treats Underactive Bladder

Background:

There is an increased prevalence of underactive bladder (UAB) symptoms with aging where the pathogenic mechanisms are unclear. Aging correlates with an overall increase in fibrosis throughout the body and may be a contributing factor to UAB. We utilized the rat model to assess aging-related bladder fibrosis and the therapeutic benefits of the antifibrotic hormone, relaxin, currently in clinical trials for treating heart failure.

Methods:

Aged (24 month) and adult (9 month) male and female F344 rats were administered recombinant human relaxin-2 (400 μg/kg/day) or saline for 14-days via subcutaneous osmotic pumps. At the end of the treatment period, bladders were isolated. Contractile function was assessed in bladder muscle strips using length-tension relationship and responses to agonists and electrical field stimulation. Bladder wall morphology was examined in histological sections stained for collagen and elastin.

Results:

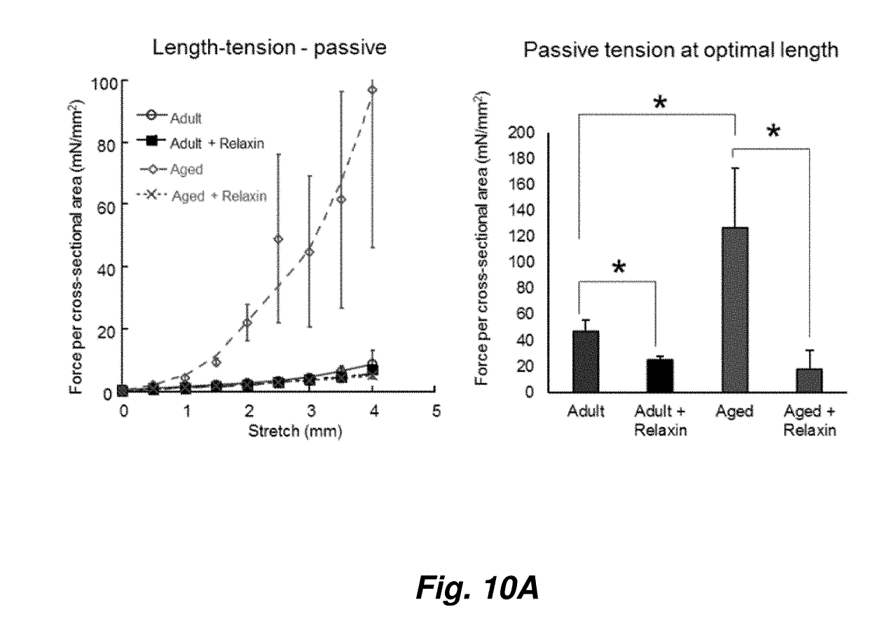

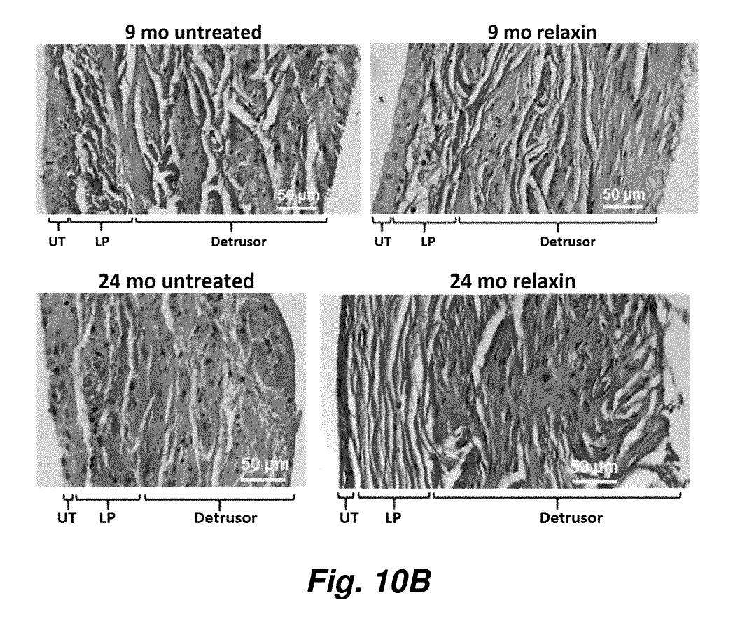

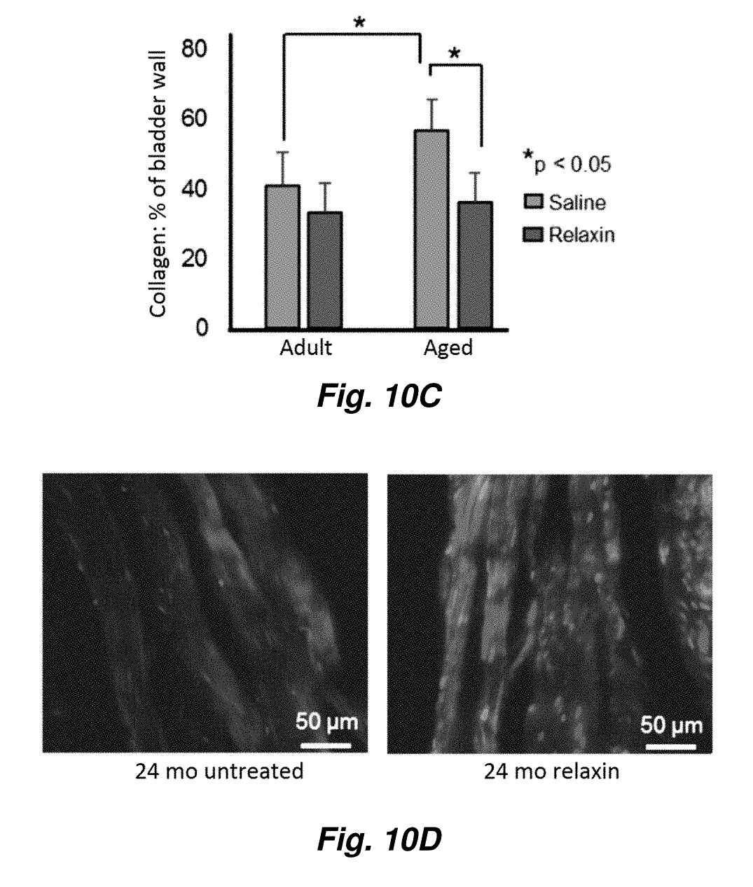

Length-tension studies demonstrated aged male rats had significantly increased passive tension compared to younger males (FIG. 10A), suggesting there is decreased bladder elasticity and compliance. Decreased compliance was not observed in aged females (not shown). Relaxin treatment of aged males showed a return of passive tension profiles comparable to adults. Histological data correlated with tissue studies, where aged males showed increased bladder collagen content which was decreased following relaxin treatment (FIG. 10B and FIG. 10C). Relaxin also increased expression of L-type Ca²⁺ channels (CaV_{1.2}) in the detrusor layer (FIG. 10D).

Discussion:

These data demonstrate a sex-specific consequence of aging, where male rats are more prone to develop bladder fibrosis than females suggesting different etiologies. We also demonstrated the therapeutic benefits of relaxin in reversing fibrosis and increasing the contractile properties of the detrusor, which represents a new therapeutic option for treating fibrosis-related UAB.

### Example 5: Relaxin Reverses Age-Related Bladder Fibrosis and Treats Underactive Bladder

Introduction:

There is an increased prevalence of underactive bladder (UAB) symptoms with aging where the pathogenic mechanisms are unclear. An overall increase in fibrosis throughout the body correlates with aging and may be a contributing factor to UAB. We utilized a rat model to assess aging-related bladder fibrosis and the therapeutic benefits of the antifibrotic hormone, human relaxin-2 (hRLX2, that is Serelaxin) (Zabbarova I, et al.: Relaxin treatment reverses age related bladder fibrosis, Neurourology & Urodynamics. 35:S176-7, 2016). Our aim was to determine the effect of aging-related fibrosis on detrusor contractility and the therapeutic benefit of hRLX2 in treating UAB.

Methods:

Aged (24 month) and adult (9 month) male and female F344 rats (obtained through the aged rodent colony of the National Institute on Aging) were administered recombinant hRLX2 (400 μg/kg/day) or saline for 14-days via subcutaneous osmotic pumps. At the end of the treatment period, bladders were isolated and contractile function assessed in organ bath experiments using muscle strips to measure length-tension relationships and responses to agonists and electrical field stimulation. Bladder wall morphology was examined in 3 μm-thick histological sections stained for collagen and elastin. The expression of the L-type Ca²⁺ channel α-subunit, Cav1.2, and relaxin receptor subtypes, RXFP1 and RXFP2, were determined by immunohistochemistry.

### Results:

Rat Bladders Express Relaxin Receptors.

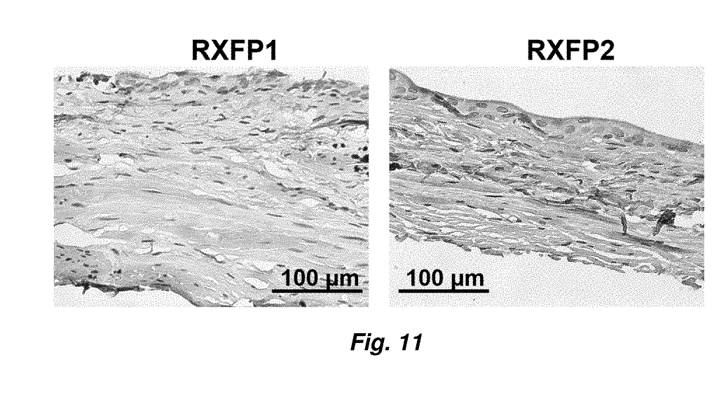

Immunohistochemical DAB staining of aged male rat bladders for RXFP1 and RXFP2 (the receptors for hRLX2) showed that RXFP2 is the dominant subtype (FIG. 11).

Relaxin Treatment Increased Detrusor Contractility and Tissue Compliance in Aged Male Rat Bladders.

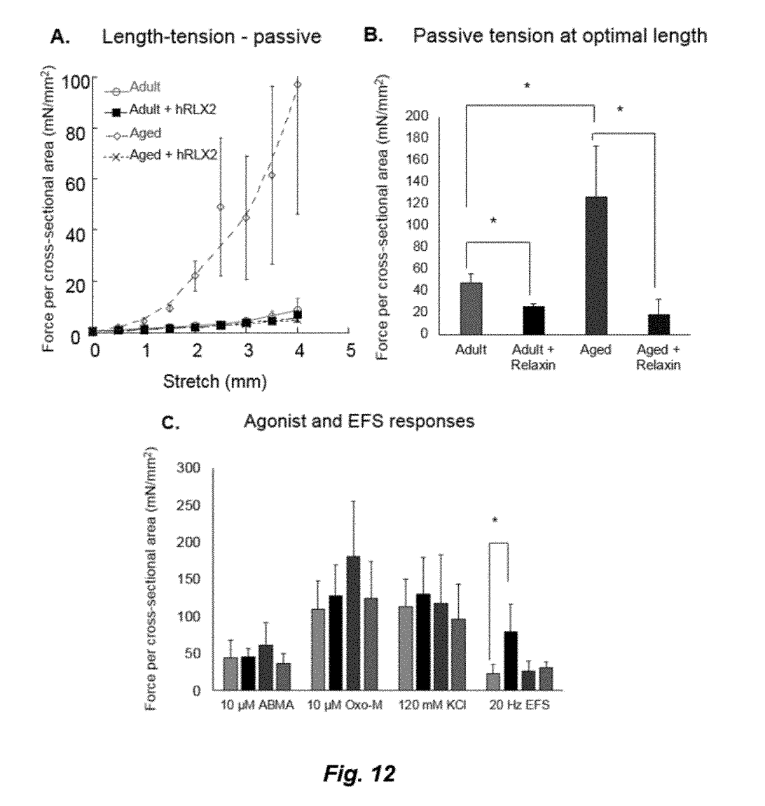

Length-tension studies demonstrated that 24 month male rats had significantly increased passive tension compared to 9 month males, suggesting decreased bladder elasticity and compliance. hRLX2 treatment of aged males showed a return of passive tension profiles comparable to adults (FIG. 12).

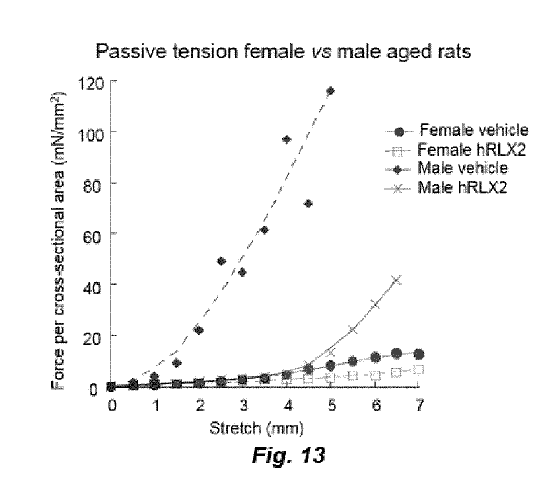

Decreased tissue compliance was not observed in aged females nor was it significantly altered by hRLX2 treatment (FIG. 13). Table 1 shows data on endogenous and exogenous relaxin plasma levels in humans (from the literature), and rats and mice.

TABLE 1
endogenous/exogenous RLX plasma levels-preliminary results

| Species | Relaxin type | Relaxin source | Plasma (pg/ml) |
|---|---|---|---|
| Human^{#} | hRLX1 | endogenous | 0.5-342 ♂ ♀ |
| Mouse | mRLX1 | endogenous | 250 ♀ |
| | | endogenous | 115 ♂ |
| Mouse | SC pump - hRLX2 | 50 μg/kg/d/14 days | 1,480 ♀ |
| | | 100 μg/kg/d/14 days | 3,081 ♀ |
| | | 400 μg/kg/d/14 days | 17,488 ♀ |
| Rat | SC pump - hRLX2 | 77 μg/kg/d/14 days | 5,042 ♂ |

^{#}human data from review of clinical trials

Aging Results in Increased Collagen Content which is Reduced by hRLX2 Therapy.

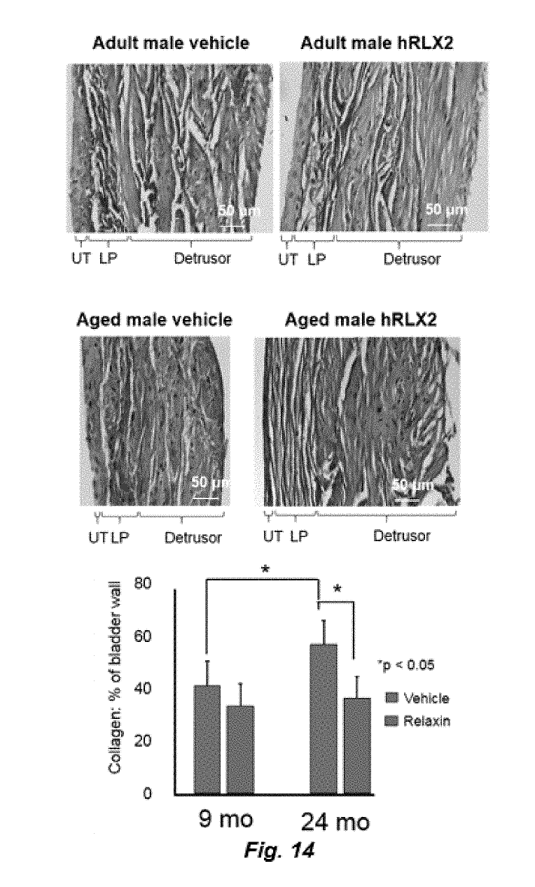

Aged male rats showed increased bladder collagen deposition (intense pink staining) which was decreased by hRLX2 treatment to levels comparable with 9 month animals (FIG. 14).

Relaxin Treatment Increases Detrusor Smooth Muscle Cav1.2 Expression.

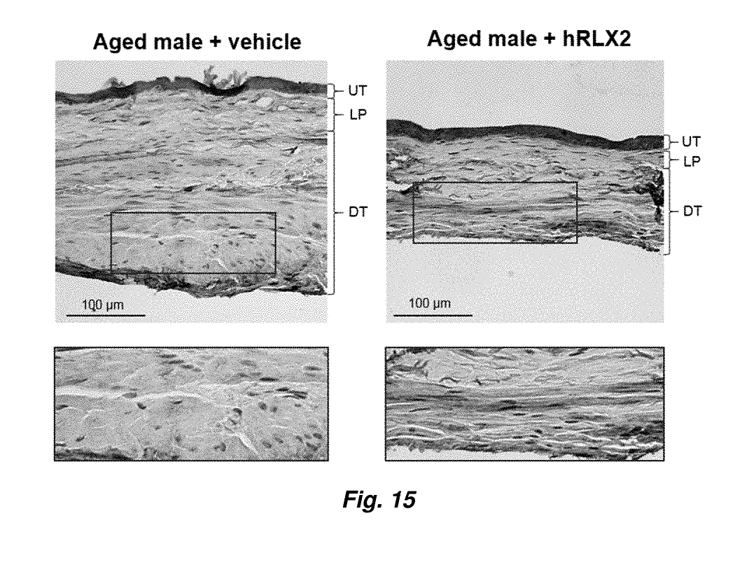

The expression of Cav1.2 in the detrusor layer was increased in aged rat bladders following hRLX2 treatment (FIG. 15). The red boxed regions highlight the increased staining for Cav1.2 in treated rats. Note: background staining of the urothelium (UT) is an artifact due to high endogenous peroxidase activity.

### Example 6: Dosing and Routes of Administration of hRLX2 for Treating LUTD

We propose that subcutaneous infusion of human relaxin-2 (hRLX2), or localized high dose injection or intravesical administration (via liposomes) to the bladder wall or prostate would be the most efficacious method for treating LUTD. As we believe that hRLX2 elicits its therapeutic effects through changes in expression of various proteins (e.g., upregulation of matrix metalloproteinases and L-type Ca²⁺ channels), a single low dose infusion of the drug may not be sufficient to initiate these processes (Jelinic, M., et al.: Short-term (48 hours) intravenous Serelaxin infusion has no effect on myogenic tone or vascular remodeling in rat mesenteric arteries. Microcirculation, 2017).

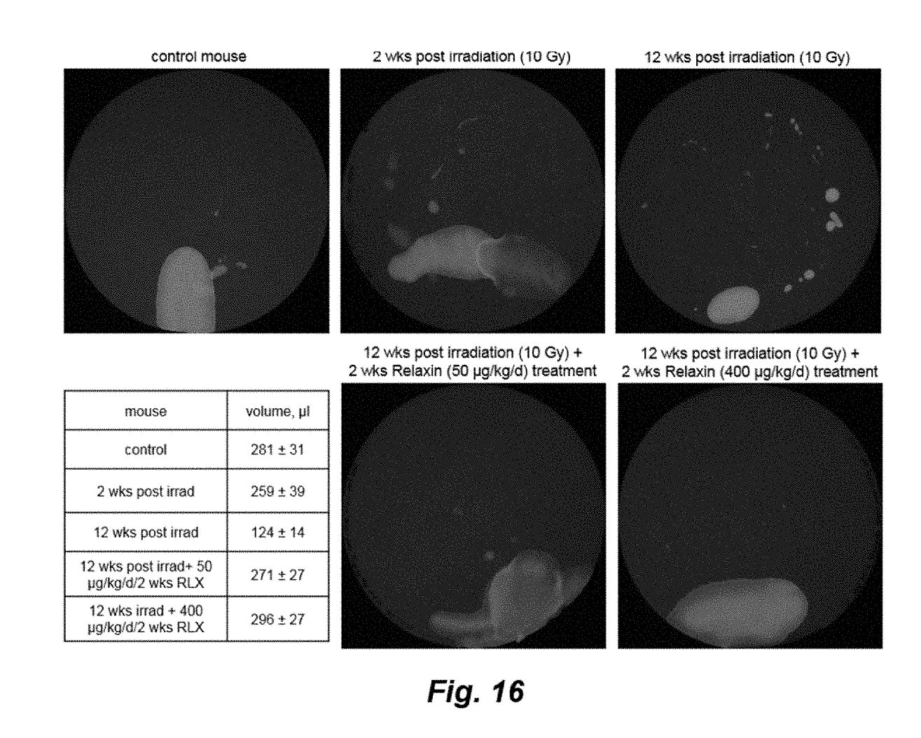

Based on our pre-clinical studies with C57Bl/6 mice, we have demonstrated that chronic radiation cystitis (via selective irradiation of the urinary bladder at a single dose of 10 Gy), causes overflow incontinence due to extensive bladder fibrosis and decreased contractile function of the detrusor smooth muscle. In vivo assessment of voiding function using urine spot test analysis (FIG. 16), unpublished data) demonstrated normal adult mice are continent and void in a single area of the cage. Chronic radiation cystitis (12 weeks post-exposure) results in multiple urine spots indicating these animals are incontinent with significantly reduced bladder capacities. A two-week treatment of irradiated mice with 50 μg/kg/day of hRLX2 (by subcutaneous osmotic pump) resulted in a small decrease in the number of spots and increase of voided volumes. However, irradiated mice treated with 400 μg/kg/day hRLX2 showed voiding pattern identical to those of non-irradiated controls, essentially reversing the damage caused by chronic radiation cystitis. Thus, our assessment is that the therapeutic dose of hRLX2 is likely between 80 and 400 μg/kg/day.

The proposed hRLX2 dosage range for treatment of LUT dysfunctions is higher than which has been utilized in clinical trials (10-250 μg/kg/day; Unemori, E.: Serelaxin in clinical development: past, present and future. Br J Pharmacol, 2016). The rationale for the higher dosages is based upon immunohistological and protein expression analysis of the hRLX2 receptors, RXFP1 and RXFP2 in the mouse bladder. We have shown that RXFP1 and 2 receptors are both expressed specifically on detrusor smooth muscle in the mouse bladder (FIG. 17), where the RXFP2 receptor is the dominant subtype unlike the heart where RXFP1 predominates. Therefore, we propose that hRLX2 exerts its effect through the RXFP2 receptor in the mouse bladder. The affinity of hRLX2 is greater for the RXFP1 than the RXFP2 receptor (Bathgate, R. A., et al.: Relaxin family peptides and their receptors. Physiol Rev, 93: 405, 2013) meaning that a higher concentration of hRLX2 would be required to activate the RXFP2 receptor. This correlates with the urine spot data described above in which higher than expected dosages were required to elicit a discernable effect. The predominance of RXFP2 receptors in the urinary bladder suggests treatment with relaxin-1 peptide or insulin-like peptide-3 (which has the highest affinity for RXFP2) may also be effective for ameliorating or reversing lower urinary tract dysfunctions.

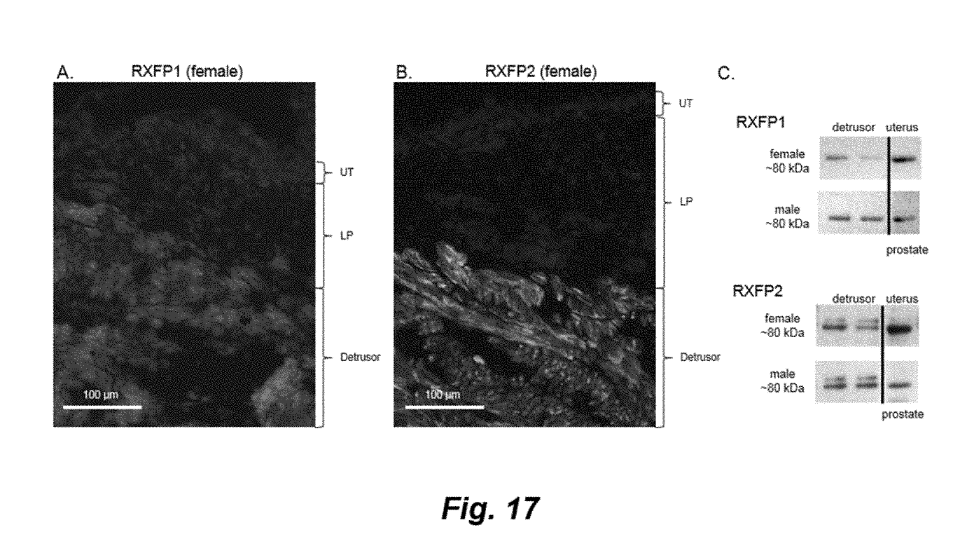

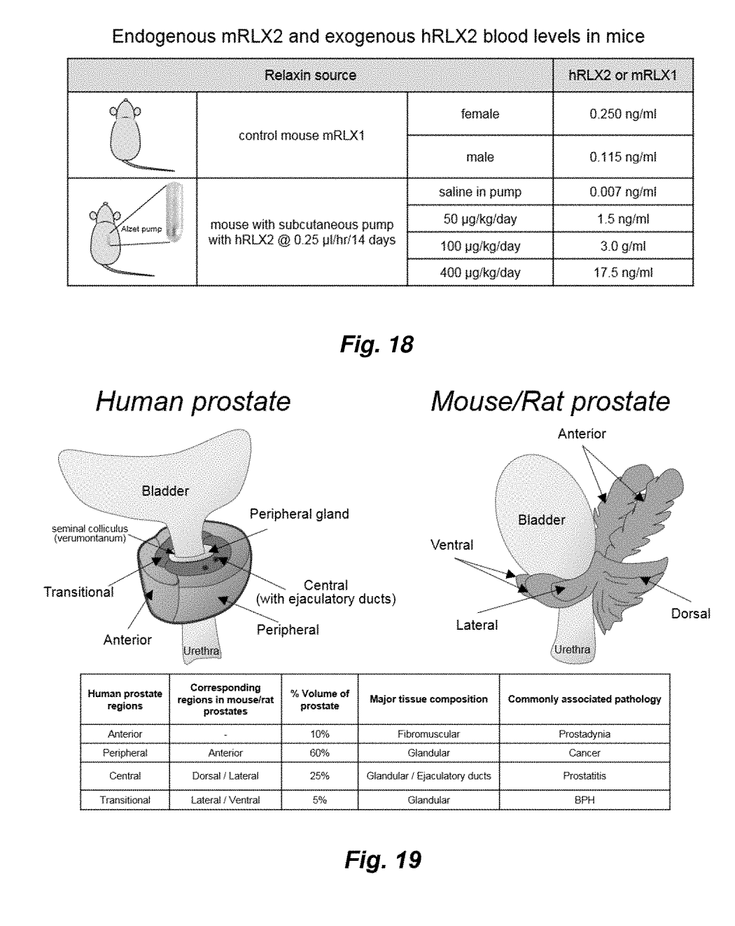

Accordingly, hRLX2 therapy for LUTD in humans could consist of subcutaneous delivery by a mini-pump of 1-400 μg/kg/day for 2 weeks to yield a level of 1-70 ng/mL in the plasma (target concentration based on mouse data shown in FIG. 18) or selective intravesical injections of hRLX2 into the bladder wall or focal injection into the prostate.

### Example 7: Benign Prostatic Hyperplasia (BPH)

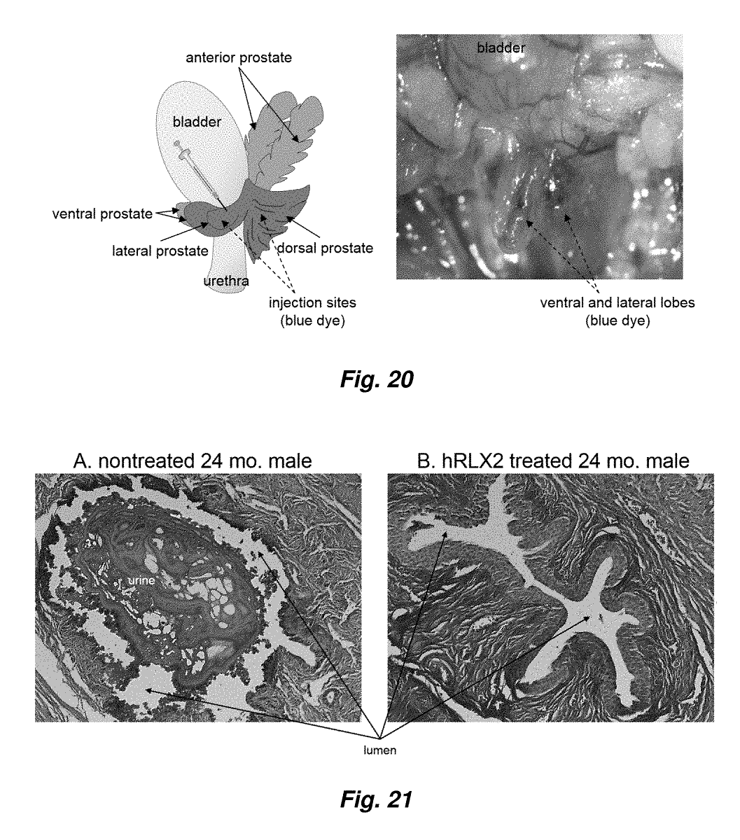

Benign Prostatic Hyperplasia is a non-malignant enlargement of the prostate and is a condition that frequently affects the elderly male population. BPH in humans can cause constriction of the urethra, outlet obstruction, inflammation, fibrosis and the development of LUTD (Bushman, W. A., et al.: The role of prostate inflammation and fibrosis in lower urinary tract symptoms. Am J Physiol Renal Physiol, 311: F817, 2016). There are considerable anatomical differences between human and rodent prostates where BPH in rodents do not cause bladder outlet obstruction (FIG. 19). However, the therapeutic effects of hRLX2 on BPH itself can still be evaluated in aged animal models. We propose that selective injection of hRLX2 (FIG. 20) to the lateral and ventral lobes (corresponding to the transitional zone of the human prostate) or systemic administration could be performed in aged rats or mice. Following treatment, the prostates could be evaluated histologically for morphological changes such as smooth muscle or extracellular matrix content, signs of inflammation (e.g., mast cell infiltration, edema) or epithelial damage, as that observed in preliminary studies (FIG. 21).

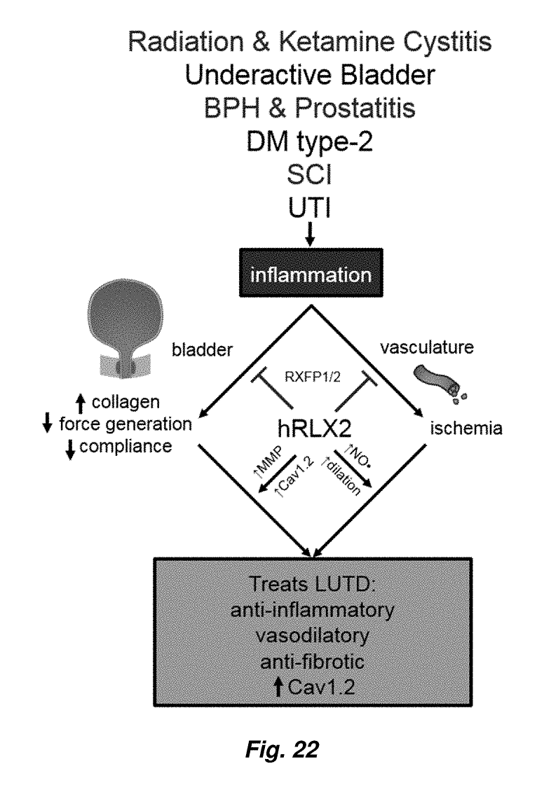

Based on the data from our lab presented here and the known safety pharmacology of recombinant human relaxin in clinical trials for heart failure and scleroderma, we claim that relaxin is a safe and effective therapy for the following pathologies: (1) Benign prostatic hyperplasia (BPH) which occurs in men with aging; radiation and chemical (e.g., cyclophosphamide and ketamine) cystitis; (3) underactive bladder (UAB) which typically occurs in women and men with aging; and (4) spinal cord injury (SCI)—as outlined in FIG. 22.

The present invention has been described with reference to certain exemplary embodiments. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments.

What is claimed is:

1. A method for treating a lower urinary tract dysfunction comprising administering to a patient in need thereof an amount of a relaxin peptide effective to treat the lower urinary tract dysfunction.

2. The method of claim 1, wherein the lower urinary tract dysfunction is a fibrosis of the bladder.

3. The method of claim 2, wherein the fibrosis is radiation-induced cystitis.

4. The method of claim 2, wherein the fibrosis is chemical-induced cystitis.

5. The method of claim 2, wherein the fibrosis is interstitial cystitis.

6. The method of claim 2, wherein the fibrosis is age-related.

7. The method of claim 1, wherein the patient is male.

8. The method of claim 1, wherein the lower urinary tract dysfunction is benign prostate hyperplasia.

9. The method of claim 1, wherein the lower urinary tract dysfunction is under active bladder.

10. The method of claim 1, wherein the lower urinary tract dysfunction is over active bladder.

11. The method of claim 1, wherein the lower urinary tract dysfunction is caused by a spinal cord injury.

12. The method of claim 1, wherein the lower urinary tract dysfunction is prostatitis.

13. The method of claim 1, wherein the lower urinary tract dysfunction is caused by diabetes mellitus.

14. The method of claim 1, wherein the lower urinary tract dysfunction is recurrent urinary tract infections and/or chronic bacterial cystitis.

15. The method of claim 1, wherein the relaxin peptide is selected from human relaxin-1, human relaxin-2, or human insulin-like peptide-3.

16. The method of claim 15, wherein the relaxin peptide is human relaxin-2 such as a human relaxin-2 peptide having a sequence of SEQ ID NO: 1, or a sequence having 80%, 90%, 95%, 96%, 97%, 98%, 99% sequence identity to SEQ ID NO: 1.

17. The method of claim 15, wherein the relaxin peptide is human insulin-like peptide-3, such as a human insulin-like peptide-3 having a sequence of SEQ ID NO: 3, or a sequence having 80%, 90%, 95%, 96%, 97%, 98%, 99% sequence identity to SEQ ID NO: 3.

18. The method of claim 1, comprising administering to the patient from 1 μg/kg/day to 400 μg/kg/day of the relaxin peptide to the patient.

19. The method of claim 1, comprising administering the relaxin peptide via a parenteral route of administration to the patient.

20. The method of claim 1, comprising administering the relaxin peptide via a catheter to the patient.


SEQUENCE LISTING (5 sequences)

SEQ ID NO: 1 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 2 - protein, 185 aa, Homo sapiens
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 3 - protein, 157 aa, Homo sapiens
MDPRLPAWALVLLGPALVFALGPAPTPEMREKLCGHHFVRALVRVCGGPRWSTEARRPATGGDQRESHSVSQAGLKLLSSSNPPTLTFQSVGISDVSCYSGWRDDICSMGWWPTVISRWDLACSPCPRPLTITATTVQLPPTLHATAASVAVPNKTC

SEQ ID NO: 4 - protein, 23 aa, Homo sapiens
QLYSALANKCCHVCTKRSLARFC

SEQ ID NO: 5 - protein, 29 aa, Homo sapiens
DSWMEEVIKLCGRELVRAQIAICGMSTWS